SEQUENCE LISTING (228 sequences)

SEQ ID NO: 1 - DNA, 18 bp, Artificial Sequence
  Description of Artificial Sequence Synthetic primer
cagacsccgaggatggcc

SEQ ID NO: 2 - DNA, 19 bp, Artificial Sequence
  Description of Artificial Sequence Synthetic primer
gcagcgaccacagctccag

SEQ ID NO: 3 - DNA, 29 bp, Artificial Sequence
  Description of Artificial Sequence Synthetic primer
accagaagtcgctgttccctyytcaggga

SEQ ID NO: 4 - DNA, 30 bp, Artificial Sequence
  Description of Artificial Sequence Synthetic primer
aaagtcacggkcccaaggctgctgcckgtg

SEQ ID NO: 5 - DNA, 24 bp, Artificial Sequence
  Description of Artificial Sequence Synthetic primer
tcacrgcagcgaccacagctccag

SEQ ID NO: 6 - DNA, 27 bp, Artificial Sequence
  Description of Artificial Sequence Synthetic primer
gcctttgcagaaacaaagtcagggttc

SEQ ID NO: 7 - DNA, 21 bp, Artificial Sequence
  Description of Artificial Sequence Synthetic primer
ccccagacsccgaggatggcc

SEQ ID NO: 8 - DNA, 33 bp, Artificial Sequence
  Description of Artificial Sequence Synthetic primer
ggaaaagtcacggkcccaaggctgctgcckgtg

SEQ ID NO: 9 - DNA, 29 bp, Artificial Sequence
  Description of Artificial Sequence Synthetic primer
cttgttctctgcttcccactcaatgtgtg

SEQ ID NO: 10 - DNA, 28 bp, Artificial Sequence
  Description of Artificial Sequence Synthetic primer
gctgagatcaggtcccatcactgccgta

SEQ ID NO: 11 - DNA, 28 bp, Artificial Sequence
  Description of Artificial Sequence Synthetic primer
gctgagatcaggtcccatcaccgctgta

SEQ ID NO: 12 - DNA, 28 bp, Artificial Sequence
  Description of Artificial Sequence Synthetic primer
gctgagatcaggtcccatcaccgccata

SEQ ID NO: 13 - DNA, 28 bp, Artificial Sequence
  Description of Artificial Sequence Synthetic primer
gctgagatcaggtcccatcaccgccgta

SEQ ID NO: 14 - DNA, 15 bp, Artificial Sequence
  Description of Artificial Sequence Synthetic primer
gggaaacsgcctctg

SEQ ID NO: 15 - DNA, 22 bp, Artificial Sequence
  Description of Artificial Sequence Synthetic primer
ggatctcggacccggagactgt

SEQ ID NO: 16 - DNA, 23 bp, Artificial Sequence
  Description of Artificial Sequence Synthetic primer
cccggtttcattttcagtttagg

SEQ ID NO: 17 - DNA, 26 bp, Artificial Sequence
  Description of Artificial Sequence Synthetic primer
attctagtgttggtcccaattgtctc

SEQ ID NO: 18 - DNA, 18 bp, Artificial Sequence
  Description of Artificial Sequence Synthetic primer
ggtgtcctgtccattctc

SEQ ID NO: 19 - DNA, 21 bp, Artificial Sequence
  Description of Artificial Sequence Synthetic primer
gagaggctcctgctttcccta

SEQ ID NO: 20 - DNA, 20 bp, Artificial Sequence
  Description of Artificial Sequence Synthetic primer
gcctctgyggggagaagcaa

SEQ ID NO: 21 - DNA, 19 bp, Artificial Sequence
  Description of Artificial Sequence Synthetic primer
cagagaggctcctgctttc

SEQ ID NO: 22 - DNA, 31 bp, Artificial Sequence
  Description of Artificial Sequence Synthetic primer
actgctaggatcaggtcccatcaccgccgta

SEQ ID NO: 23 - DNA, 31 bp, Artificial Sequence
  Description of Artificial Sequence Synthetic primer
actgctaggatcaggtcccatcaccgccata

SEQ ID NO: 24 - DNA, 31 bp, Artificial Sequence
  Description of Artificial Sequence Synthetic primer
actgctaggatcaggtcccatcaccgctgta

SEQ ID NO: 25 - DNA, 31 bp, Artificial Sequence
  Description of Artificial Sequence Synthetic primer
actgctaggatcaggtcccatcactgccgta

SEQ ID NO: 26 - DNA, 34 bp, Artificial Sequence
  Description of Artificial Sequence Synthetic primer
caggccttgttctctgcttcacactcaatgtgtg

SEQ ID NO: 27 - DNA, 29 bp, Artificial Sequence
  Description of Artificial Sequence Synthetic primer
cagggccttaaggtcctagaggaacctcc

SEQ ID NO: 28 - DNA, 31 bp, Artificial Sequence
  Description of Artificial Sequence Synthetic primer
gaacctggtcagatcccacagaasatgtggc

SEQ ID NO: 29 - DNA, 24 bp, Artificial Sequence
  Description of Artificial Sequence Synthetic primer
tgggtgagctcccccatgggctcc

SEQ ID NO: 30 - DNA, 24 bp, Artificial Sequence
  Description of Artificial Sequence Synthetic primer
tgggtgggctcccccatgggctcc

SEQ ID NO: 31 - DNA, 24 bp, Artificial Sequence
  Description of Artificial Sequence Synthetic primer
tggttgagctcccccatgggctcc

SEQ ID NO: 32 - DNA, 24 bp, Artificial Sequence
  Description of Artificial Sequence Synthetic primer
tgggtgagctcccccacgggctcc

SEQ ID NO: 33 - DNA, 33 bp, Artificial Sequence
  Description of Artificial Sequence Synthetic primer
ggaaaagtcacgggcccaaggctgctgcckgtg

SEQ ID NO: 34 - DNA, 30 bp, Artificial Sequence
  Description of Artificial Sequence Synthetic primer
caggtgcctttgcagaaacaaagtcagggt

SEQ ID NO: 35 - DNA, 29 bp, Artificial Sequence
  Description of Artificial Sequence Synthetic primer
cacggaatagragattatcccaggtgcct

SEQ ID NO: 36 - DNA, 23 bp, Artificial Sequence
  Description of Artificial Sequence Synthetic primer
ggtkcccaaggctgctgcagggg

SEQ ID NO: 37 - DNA, 22 bp, Artificial Sequence
  Description of Artificial Sequence Synthetic primer
gaaccgtcctcctgctgctctc

SEQ ID NO: 38 - DNA, 22 bp, Artificial Sequence
  Description of Artificial Sequence Synthetic primer
gaaccgtcctcctgctgctctg

SEQ ID NO: 39 - DNA, 26 bp, Artificial Sequence
  Description of Artificial Sequence Synthetic primer
atcacagcagcgaccacagctccgat

SEQ ID NO: 40 - DNA, 26 bp, Artificial Sequence
  Description of Artificial Sequence Synthetic primer
atcacagtagcgaccacagctccgat

SEQ ID NO: 41 - DNA, 26 bp, Artificial Sequence
  Description of Artificial Sequence Synthetic primer
atcacagtagcaaccacagctccgat

SEQ ID NO: 42 - DNA, 26 bp, Artificial Sequence
  Description of Artificial Sequence Synthetic primer
atcacagcagcgaccacagcgaccac

SEQ ID NO: 43 - DNA, 33 bp, Artificial Sequence
  Description of Artificial Sequence Synthetic primer
ggctctgattccagcacttctgagtcactttac

SEQ ID NO: 44 - DNA, 27 bp, Artificial Sequence
  Description of Artificial Sequence Synthetic primer
gaccacaggctggggcgcaggacccgg

SEQ ID NO: 45 - DNA, 27 bp, Artificial Sequence
  Description of Artificial Sequence Synthetic primer
gaccacaggcgggggcgcaggacctga

SEQ ID NO: 46 - DNA, 22 bp, Artificial Sequence
  Description of Artificial Sequence Synthetic primer
acgcacccacccggactcagaa

SEQ ID NO: 47 - DNA, 22 bp, Artificial Sequence
  Description of Artificial Sequence Synthetic primer
acgcacccacccggactcagag

SEQ ID NO: 48 - DNA, 26 bp, Artificial Sequence
  Description of Artificial Sequence Synthetic primer
agaggctcttgaagtcacaaagggga

SEQ ID NO: 49 - DNA, 27 bp, Artificial Sequence
  Description of Artificial Sequence Synthetic primer
actgtgaaccgtcctcctgctgctctc

SEQ ID NO: 50 - DNA, 28 bp, Artificial Sequence
  Description of Artificial Sequence Synthetic primer
aagtgcgaaccctcctcctgctgctctg

SEQ ID NO: 51 - DNA, 28 bp, Artificial Sequence
  Description of Artificial Sequence Synthetic primer
aagtgcgaaccgtcctcctgctgctctg

SEQ ID NO: 52 - DNA, 28 bp, Artificial Sequence
  Description of Artificial Sequence Synthetic primer
actgcggtkcccaaggctgctgcagggg

SEQ ID NO: 53 - DNA, 29 bp, Artificial Sequence
  Description of Artificial Sequence Synthetic primer
attatgattaagcccctcctcrcccccag

SEQ ID NO: 54 - DNA, 29 bp, Artificial Sequence
  Description of Artificial Sequence Synthetic primer
attatgattacagcccctccttgccccag

SEQ ID NO: 55 - DNA, 29 bp, Artificial Sequence
  Description of Artificial Sequence Synthetic primer
attatgattaagcccctcctggcccccag

SEQ ID NO: 56 - DNA, 19 bp, Artificial Sequence
  Description of Artificial Sequence Synthetic primer
ggaggggtcgtgacctgcg

SEQ ID NO: 57 - DNA, 26 bp, Artificial Sequence
  Description of Artificial Sequence Synthetic primer
attatgattaggggacggggctgacc

SEQ ID NO: 58 - DNA, 26 bp, Artificial Sequence
  Description of Artificial Sequence Synthetic primer
attatgattaggggactgggctgacc

SEQ ID NO: 59 - DNA, 26 bp, Artificial Sequence
  Description of Artificial Sequence Synthetic primer
attatgattaggggacggtgctgacc

SEQ ID NO: 60 - DNA, 22 bp, Artificial Sequence
  Description of Artificial Sequence Synthetic primer
aaactcatgccattctccattc

SEQ ID NO: 61 - DNA, 18 bp, Artificial Sequence
  Description of Artificial Sequence Synthetic primer
gtcacatgggtggtccta

SEQ ID NO: 62 - DNA, 21 bp, Artificial Sequence
  Description of Artificial Sequence Synthetic primer
ggctcctgctttccctgagaa

SEQ ID NO: 63 - DNA, 27 bp, Artificial Sequence
  Description of Artificial Sequence Synthetic primer
attatgattacccctcctcrcccccag

SEQ ID NO: 64 - DNA, 27 bp, Artificial Sequence
  Description of Artificial Sequence Synthetic primer
attatgattagcccctccttgccccag

SEQ ID NO: 65 - DNA, 27 bp, Artificial Sequence
  Description of Artificial Sequence Synthetic primer
attatgattacccctcctggcccccag

SEQ ID NO: 66 - DNA, 27 bp, Artificial Sequence
  Description of Artificial Sequence Synthetic primer
attatgattacccctcctcgctcccag

SEQ ID NO: 67 - DNA, 25 bp, Artificial Sequence
  Description of Artificial Sequence Synthetic primer
attatgattacctcctcrcccccag

SEQ ID NO: 68 - DNA, 25 bp, Artificial Sequence
  Description of Artificial Sequence Synthetic primer
attatgattaccctccttgccccag

SEQ ID NO: 69 - DNA, 25 bp, Artificial Sequence
  Description of Artificial Sequence Synthetic primer
attatgattacctcctggcccccag

SEQ ID NO: 70 - DNA, 25 bp, Artificial Sequence
  Description of Artificial Sequence Synthetic primer
attatgattacctcctcgctcccag

SEQ ID NO: 71 - DNA, 19 bp, Artificial Sequence
  Description of Artificial Sequence Synthetic primer
cagcccctccttgccccag

SEQ ID NO: 72 - DNA, 19 bp, Artificial Sequence
  Description of Artificial Sequence Synthetic primer
agcccctcctcrcccccag

SEQ ID NO: 73 - DNA, 19 bp, Artificial Sequence
  Description of Artificial Sequence Synthetic primer
agctcctcctcgcccccag

SEQ ID NO: 74 - DNA, 19 bp, Artificial Sequence
  Description of Artificial Sequence Synthetic primer
agcccctcctggcccccag

SEQ ID NO: 75 - DNA, 16 bp, Artificial Sequence
  Description of Artificial Sequence Synthetic primer
ggggacggggctgacc

SEQ ID NO: 76 - DNA, 16 bp, Artificial Sequence
  Description of Artificial Sequence Synthetic primer
ggggactgggctgacc

SEQ ID NO: 77 - DNA, 16 bp, Artificial Sequence
  Description of Artificial Sequence Synthetic primer
ggggacggtgctgacc

SEQ ID NO: 78 - DNA, 19 bp, Artificial Sequence
  Description of Artificial Sequence Synthetic primer
gcagtggtcaaagtggtca

SEQ ID NO: 79 - DNA, 20 bp, Artificial Sequence
  Description of Artificial Sequence Synthetic primer
gcagctgtggtcaggctgct

SEQ ID NO: 80 - DNA, 24 bp, Artificial Sequence
  Description of Artificial Sequence Synthetic primer
ggacacgggggtgrgctgtctstc

SEQ ID NO: 81 - DNA, 25 bp, Artificial Sequence
  Description of Artificial Sequence Synthetic primer
cagtcccggttctgaagtccccagt

SEQ ID NO: 82 - DNA, 25 bp, Artificial Sequence
  Description of Artificial Sequence Synthetic primer
cagtcccggttctaaagtccccagt

SEQ ID NO: 83 - DNA, 20 bp, Artificial Sequence
  Description of Artificial Sequence Synthetic primer
gggccggtgagtgcggggtt

SEQ ID NO: 84 - DNA, 20 bp, Artificial Sequence
  Description of Artificial Sequence Synthetic primer
gggcctgtgagtgcgaggtt

SEQ ID NO: 85 - DNA, 20 bp, Artificial Sequence
  Description of Artificial Sequence Synthetic primer
gggcctgtgagtgcggggtt

SEQ ID NO: 86 - DNA, 22 bp, Artificial Sequence
  Description of Artificial Sequence Synthetic primer
agctccaaggacagctaggaca

SEQ ID NO: 87 - DNA, 22 bp, Artificial Sequence
  Description of Artificial Sequence Synthetic primer
agctcctaggacagctaggaca

SEQ ID NO: 88 - DNA, 22 bp, Artificial Sequence
  Description of Artificial Sequence Synthetic primer
gacagccaggacagccaggaca

SEQ ID NO: 89 - DNA, 21 bp, Artificial Sequence
  Description of Artificial Sequence Synthetic primer
gtgaggggccctgacctccaa

SEQ ID NO: 90 - DNA, 21 bp, Artificial Sequence
  Description of Artificial Sequence Synthetic primer
gtgaggggccctgacccccaa

SEQ ID NO: 91 - DNA, 21 bp, Artificial Sequence
  Description of Artificial Sequence Synthetic primer
gtgaggggcccttacacccaa

SEQ ID NO: 92 - DNA, 25 bp, Artificial Sequence
  Description of Artificial Sequence Synthetic primer
gccatcacagctcctaggacagcta

SEQ ID NO: 93 - DNA, 25 bp, Artificial Sequence
  Description of Artificial Sequence Synthetic primer
gccaccatagctcctaggacagcta

SEQ ID NO: 94 - DNA, 25 bp, Artificial Sequence
  Description of Artificial Sequence Synthetic primer
gtgaccacagctccaaggacagcta

SEQ ID NO: 95 - DNA, 23 bp, Artificial Sequence
  Description of Artificial Sequence Synthetic primer
agctaggacagccaggacagcca

SEQ ID NO: 96 - DNA, 24 bp, Artificial Sequence
  Description of Artificial Sequence Synthetic primer
ccaccacagctcctaggacagcta

SEQ ID NO: 97 - DNA, 25 bp, Artificial Sequence
  Description of Artificial Sequence Synthetic primer
cagtcccggttctraagtccccagt

SEQ ID NO: 98 - DNA, 25 bp, Artificial Sequence
  Description of Artificial Sequence Synthetic primer
ccactcccattgggtgtcggrttct

SEQ ID NO: 99 - DNA, 29 bp, Artificial Sequence
  Description of Artificial Sequence Synthetic primer
ccacagctgcygcagtagtcaaagtggtc

SEQ ID NO: 100 - DNA, 30 bp, Artificial Sequence
  Description of Artificial Sequence Synthetic primer
ctcaggtcaggaccagaagtcgctgttcat

SEQ ID NO: 101 - DNA, 24 bp, Artificial Sequence
  Description of Artificial Sequence Synthetic primer
ctgagatggcccaggtgtggatgg

SEQ ID NO: 102 - DNA, 24 bp, Artificial Sequence
  Description of Artificial Sequence Synthetic primer
ctgagatggcccatgtgtggatgg

SEQ ID NO: 103 - DNA, 17 bp, Artificial Sequence
  Description of Artificial Sequence Synthetic primer
ggagccgcgcagggagg

SEQ ID NO: 104 - DNA, 18 bp, Artificial Sequence
  Description of Artificial Sequence Synthetic primer
gggtcgggcgggtctcag

SEQ ID NO: 105 - DNA, 18 bp, Artificial Sequence
  Description of Artificial Sequence Synthetic primer
ggccgtccgtgggggatg

SEQ ID NO: 106 - DNA, 17 bp, Artificial Sequence
  Description of Artificial Sequence Synthetic primer
tcgkgacctgcgccccg

SEQ ID NO: 107 - DNA, 24 bp, Artificial Sequence
  Description of Artificial Sequence Synthetic primer
ttcrgtttaggccaaaatccccgc

SEQ ID NO: 108 - DNA, 25 bp, Artificial Sequence
  Description of Artificial Sequence Synthetic primer
gtcrcctttacccggtttcattttc

SEQ ID NO: 109 - DNA, 24 bp, Artificial Sequence
  Description of Artificial Sequence Synthetic primer
gctgatcccattttcctcccctcc

SEQ ID NO: 110 - DNA, 22 bp, Artificial Sequence
  Description of Artificial Sequence Synthetic primer
aggctggcgtctgggttctgtg

SEQ ID NO: 111 - DNA, 25 bp, Artificial Sequence
  Description of Artificial Sequence Synthetic primer
ccrttctcaggatrgtcacatgggc

SEQ ID NO: 112 - DNA, 29 bp, Artificial Sequence
  Description of Artificial Sequence Synthetic primer
caaagtgtctgaattttctgactcttccc

SEQ ID NO: 113 - DNA, 24 bp, Artificial Sequence
  Description of Artificial Sequence Synthetic primer
aggacttctgctttcyctgakaag

SEQ ID NO: 114 - DNA, 32 bp, Artificial Sequence
  Description of Artificial Sequence Synthetic primer
atgatattatgattaggagccgcgcagggagg

SEQ ID NO: 115 - DNA, 30 bp, Artificial Sequence
  Description of Artificial Sequence Synthetic primer
attatgattactcgggggacggggctgacc

SEQ ID NO: 116 - DNA, 27 bp, Artificial Sequence
  Description of Artificial Sequence Synthetic primer
atgattaacccctcatccccctcctta

SEQ ID NO: 117 - DNA, 27 bp, Artificial Sequence
  Description of Artificial Sequence Synthetic primer
atgattaaccccccattcccctcctta

SEQ ID NO: 118 - DNA, 35 bp, Artificial Sequence
  Description of Artificial Sequence Synthetic primer
atgatattatgattaacccctcatccccctcctta

SEQ ID NO: 119 - DNA, 35 bp, Artificial Sequence
  Description of Artificial Sequence Synthetic primer
atgatattatgattaaccccccattcccctcctta

SEQ ID NO: 120 - DNA, 38 bp, Artificial Sequence
  Description of Artificial Sequence Synthetic primer
tgtaaaacgacggccagtcccacagcacgtttcttgtg

SEQ ID NO: 121 - DNA, 37 bp, Artificial Sequence
  Description of Artificial Sequence Synthetic primer
tgtaaaacgacggccagtcccacagcacgtttcctgt

SEQ ID NO: 122 - DNA, 42 bp, Artificial Sequence
  Description of Artificial Sequence Synthetic primer
tgtaaaacgacggccagtttcacagcacgtttcttggagtac

SEQ ID NO: 123 - DNA, 44 bp, Artificial Sequence
  Description of Artificial Sequence Synthetic primer
tgtaaaacgacggccagttactaatcacgtttcttggagcaggt

SEQ ID NO: 124 - DNA, 37 bp, Artificial Sequence
  Description of Artificial Sequence Synthetic primer
tgtaaaacgacggccagttccacagcacgtttcttga

SEQ ID NO: 125 - DNA, 43 bp, Artificial Sequence
  Description of Artificial Sequence Synthetic primer
tgtaaaacgacggccagttactaatcacgtttcttggaggagg

SEQ ID NO: 126 - DNA, 49 bp, Artificial Sequence
  Description of Artificial Sequence Synthetic primer
tgtaaaacgacggccagttactaatcacgtttcttggagcaggttaaac

SEQ ID NO: 127 - DNA, 40 bp, Artificial Sequence
  Description of Artificial Sequence Synthetic primer
tgtaaaacgacggccagtatcacagcacgtttcttggagg

SEQ ID NO: 128 - DNA, 50 bp, Artificial Sequence
  Description of Artificial Sequence Synthetic primer
tgtaaaacgacggccagttactaatcacgtttcttgaagcaggataagtt

SEQ ID NO: 129 - DNA, 37 bp, Artificial Sequence
  Description of Artificial Sequence Synthetic primer
caggaaacagctatgacccrygctyacctcgcckctg

SEQ ID NO: 130 - DNA, 49 bp, Artificial Sequence
  Description of Artificial Sequence Synthetic primer
tgtaaacgacggccagttactaattgtgtttcttgaagcaggataagtt

SEQ ID NO: 131 - DNA, 18 bp, Artificial Sequence
  Description of Artificial Sequence Synthetic primer
tgtaaaacgacggccagt

SEQ ID NO: 132 - DNA, 18 bp, Artificial Sequence
  Description of Artificial Sequence Synthetic primer
caggaaacagctatgacc

SEQ ID NO: 133 - DNA, 38 bp, Artificial Sequence
  Description of Artificial Sequence Synthetic primer
tgtaaaacgacggccagtcacgtttcttgtggsagctt

SEQ ID NO: 134 - DNA, 37 bp, Artificial Sequence
  Description of Artificial Sequence Synthetic primer
tgtaaaacgacggccagtttcctgtggcagcctaaga

SEQ ID NO: 135 - DNA, 41 bp, Artificial Sequence
  Description of Artificial Sequence Synthetic primer
tgtaaaacgacggccagtcgtttcttggagtactctacgtc

SEQ ID NO: 136 - DNA, 40 bp, Artificial Sequence
  Description of Artificial Sequence Synthetic primer
tgtaaaacgacggccagtcgtttcttggagcaggttaaac

SEQ ID NO: 137 - DNA, 40 bp, Artificial Sequence
  Description of Artificial Sequence Synthetic primer
tgtaaaacgacggccagtttcctgtggcagggtaagtata

SEQ ID NO: 138 - DNA, 41 bp, Artificial Sequence
  Description of Artificial Sequence Synthetic primer
tgtaaaacgacggccagtcgtttcttggagtactctabggg

SEQ ID NO: 139 - DNA, 39 bp, Artificial Sequence
  Description of Artificial Sequence Synthetic primer
tgtaaaacgacggccagttttcttggagtactctabggg

SEQ ID NO: 140 - DNA, 41 bp, Artificial Sequence
  Description of Artificial Sequence Synthetic primer
tgtaaaacgacggccagtgtttcttggagtactctabgggt

SEQ ID NO: 141 - DNA, 40 bp, Artificial Sequence
  Description of Artificial Sequence Synthetic primer
tgtaaaacgacggccagttttcttggagtactctabgggt

SEQ ID NO: 142 - DNA, 40 bp, Artificial Sequence
  Description of Artificial Sequence Synthetic primer
tgtaaaacgacggccagtgtttcttgaagcaggataagtt

SEQ ID NO: 143 - DNA, 38 bp, Artificial Sequence
  Description of Artificial Sequence Synthetic primer
tgtaaaacgacggccagtcacagcacgtttcttggagg

SEQ ID NO: 144 - DNA, 38 bp, Artificial Sequence
  Description of Artificial Sequence Synthetic primer
tgtaaaacgacggccagtgsagctgyktaagtctgagt

SEQ ID NO: 145 - DNA, 38 bp, Artificial Sequence
  Description of Artificial Sequence Synthetic primer
tgtaaaacgacggccagtagcgagtgtggaacctgatc

SEQ ID NO: 146 - DNA, 36 bp, Artificial Sequence
  Description of Artificial Sequence Synthetic primer
tgtaaaacgacggccagtgcagcaggataagtatga

SEQ ID NO: 147 - DNA, 37 bp, Artificial Sequence
  Description of Artificial Sequence Synthetic primer
caggaaacagctatgaccgctyacctcgcckctgcac

SEQ ID NO: 148 - DNA, 20 bp, Artificial Sequence
  Description of Artificial Sequence Synthetic primer
tcatgcttttggccagacag

SEQ ID NO: 149 - DNA, 19 bp, Artificial Sequence
  Description of Artificial Sequence Synthetic primer
ggcggactcccagcttgta

SEQ ID NO: 150 - DNA, 20 bp, Artificial Sequence
  Description of Artificial Sequence Synthetic primer
ctgcacygtgaakctctcca

SEQ ID NO: 151 - DNA, 19 bp, Artificial Sequence
  Description of Artificial Sequence Synthetic primer
gcacygtgaakctctccac

SEQ ID NO: 152 - DNA, 19 bp, Artificial Sequence
  Description of Artificial Sequence Synthetic primer
gcacygtgaagctctcacc

SEQ ID NO: 153 - DNA, 33 bp, Artificial Sequence
  Description of Artificial Sequence Synthetic primer
ttttttttttttttgcacygtgaagctcttacc

SEQ ID NO: 154 - DNA, 33 bp, Artificial Sequence
  Description of Artificial Sequence Synthetic primer
ttttttttttttttgtacygtgaakctccccac

SEQ ID NO: 155 - DNA, 33 bp, Artificial Sequence
  Description of Artificial Sequence Synthetic primer
ttttttttttttttgcacygtgaakctccccac

SEQ ID NO: 156 - DNA, 33 bp, Artificial Sequence
  Description of Artificial Sequence Synthetic primer
ttttttttttttttgtacygtgaakctcaccac

SEQ ID NO: 157 - DNA, 33 bp, Artificial Sequence
  Description of Artificial Sequence Synthetic primer
ttttttttttttttgcacygtgaakctcaccac

SEQ ID NO: 158 - DNA, 19 bp, Artificial Sequence
  Description of Artificial Sequence Synthetic primer
ctgtggcagggtaagtata

SEQ ID NO: 159 - DNA, 19 bp, Artificial Sequence
  Description of Artificial Sequence Synthetic primer
ttcttggagcaggttaaac

SEQ ID NO: 160 - DNA, 17 bp, Artificial Sequence
  Description of Artificial Sequence Synthetic primer
cctgtggcagcctaaga

SEQ ID NO: 161 - DNA, 18 bp, Artificial Sequence
  Description of Artificial Sequence Synthetic primer
cgtttcttgtggsagctt

SEQ ID NO: 162 - DNA, 20 bp, Artificial Sequence
  Description of Artificial Sequence Synthetic primer
ttcttggagtactctacgtc

SEQ ID NO: 163 - DNA, 19 bp, Artificial Sequence
  Description of Artificial Sequence Synthetic primer
ccacagcacgtttcttgtg

SEQ ID NO: 164 - DNA, 23 bp, Artificial Sequence
  Description of Artificial Sequence Synthetic primer
cgtttcttggagtactctacggg

SEQ ID NO: 165 - DNA, 40 bp, Artificial Sequence
  Description of Artificial Sequence Synthetic primer
tgtaaaacgacggccagtcctccccgcagagaattamgtg

SEQ ID NO: 166 - DNA, 40 bp, Artificial Sequence
  Description of Artificial Sequence Synthetic primer
tgtaaaacgacggccagtcctccccgcagagaattacctt

SEQ ID NO: 167 - DNA, 38 bp, Artificial Sequence
  Description of Artificial Sequence Synthetic primer
caggaaacagctatgaccgcgctgyagggtcacggcct

SEQ ID NO: 168 - DNA, 38 bp, Artificial Sequence
  Description of Artificial Sequence Synthetic primer
caggaaacagctatgaccgcgctgcagggtcatgggcc

SEQ ID NO: 169 - DNA, 38 bp, Artificial Sequence
  Description of Artificial Sequence Synthetic primer
tgtaaaacgacggccagtggtgattccccgcagaggat

SEQ ID NO: 170 - DNA, 39 bp, Artificial Sequence
  Description of Artificial Sequence Synthetic primer
caggaaacagctatgaccgggcctcgcagasgggcgacg

SEQ ID NO: 171 - DNA, 39 bp, Artificial Sequence
  Description of Artificial Sequence Synthetic primer
caggaaacagctatgaccgsgcctcacggaggggcgacg

SEQ ID NO: 172 - DNA, 39 bp, Artificial Sequence
  Description of Artificial Sequence Synthetic primer
caggaaacagctatgaccgcgcctcacggagggtcaacc

SEQ ID NO: 173 - DNA, 44 bp, Artificial Sequence
  Description of Artificial Sequence Synthetic primer
cagtcgaggctgatagcgagctccctgtctgttactgccctyag

SEQ ID NO: 174 - DNA, 44 bp, Artificial Sequence
  Description of Artificial Sequence Synthetic primer
ctatcaacaggttgaactgggcccacagtaacagaaactcaata

SEQ ID NO: 175 - DNA, 44 bp, Artificial Sequence
  Description of Artificial Sequence Synthetic primer
ctatcaacaggttgaactgggcccataataacagaaactcaata

SEQ ID NO: 176 - DNA, 38 bp, Artificial Sequence
  Description of Artificial Sequence Synthetic primer
caggaaacagctatgaccactgactggccggtgattcc

SEQ ID NO: 177 - DNA, 38 bp, Artificial Sequence
  Description of Artificial Sequence Synthetic primer
caggaaacagctatgaccactgaccggccggtgattcc

SEQ ID NO: 178 - DNA, 41 bp, Artificial Sequence
  Description of Artificial Sequence Synthetic primer
gtaaaacgacggccagtatgggcctcgcagacgggcgacga

SEQ ID NO: 179 - DNA, 38 bp, Artificial Sequence
  Description of Artificial Sequence Synthetic primer
caggaaacagctatgacccctgcccccaccactctcgc

SEQ ID NO: 180 - DNA, 39 bp, Artificial Sequence
  Description of Artificial Sequence Synthetic primer
caggaaacagctatgaccgacactaggcagcctggccaa

SEQ ID NO: 181 - DNA, 41 bp, Artificial Sequence
  Description of Artificial Sequence Synthetic primer
caggaaacagctatgacccagagcagaggacaaggccgacg

SEQ ID NO: 182 - DNA, 44 bp, Artificial Sequence
  Description of Artificial Sequence Synthetic primer
caggaaacagctatgaccaaaaggaggcaaatgcataaggcacg

SEQ ID NO: 183 - DNA, 40 bp, Artificial Sequence
  Description of Artificial Sequence Synthetic primer
caggaaacagctatgaccgcgcctcacggaggggcgacga

SEQ ID NO: 184 - DNA, 39 bp, Artificial Sequence
  Description of Artificial Sequence Synthetic primer
gtaaaacgacggccagtgggcctcgcagaggggcgacgc

SEQ ID NO: 185 - DNA, 19 bp, Artificial Sequence
  Description of Artificial Sequence Synthetic primer
ctatcaacaggttgaactg

SEQ ID NO: 186 - DNA, 22 bp, Artificial Sequence
  Description of Artificial Sequence Synthetic primer
cagtcgaggctgatagcgagct

SEQ ID NO: 187 - DNA, 39 bp, Artificial Sequence
  Description of Artificial Sequence Synthetic primer
gtaaaacgacggccagtgcgtgcgtcttgtgagcagaag

SEQ ID NO: 188 - DNA, 39 bp, Artificial Sequence
  Description of Artificial Sequence Synthetic primer
gtaaaacgacggccagtgtgctacttcaccaacgggagg

SEQ ID NO: 189 - DNA, 39 bp, Artificial Sequence
  Description of Artificial Sequence Synthetic primer
gtaaaacgacggccagtgtgctacttcaccaacgggagc

SEQ ID NO: 190 - DNA, 36 bp, Artificial Sequence
  Description of Artificial Sequence Synthetic primer
caggaaacagctatgacctcgccgctgcaaggtcgt

SEQ ID NO: 191 - DNA, 42 bp, Artificial Sequence
  Description of Artificial Sequence Synthetic primer
gtaaaacgacggccagtgatttcgtgtaccagtttaagggtc

SEQ ID NO: 192 - DNA, 44 bp, Artificial Sequence
  Description of Artificial Sequence Synthetic primer
gtaaaacgacggccagtaggatttcgtgtaccagtttaagggta

SEQ ID NO: 193 - DNA, 44 bp, Artificial Sequence
  Description of Artificial Sequence Synthetic primer
gtaaaacgacggccagtaggatttcgtgttccagtttaagggta

SEQ ID NO: 194 - DNA, 44 bp, Artificial Sequence
  Description of Artificial Sequence Synthetic primer
gtaaaacgacggccagtaggatttcgtgttccagtttaaggcta

SEQ ID NO: 195 - DNA, 36 bp, Artificial Sequence
  Description of Artificial Sequence Synthetic primer
caggaaacagctatgacctctcctctgcaagatccc

SEQ ID NO: 196 - DNA, 36 bp, Artificial Sequence
  Description of Artificial Sequence Synthetic primer
caggaaacagctatgacctctcctctgcaggatccc

SEQ ID NO: 197 - DNA, 20 bp, Artificial Sequence
  Description of Artificial Sequence Synthetic primer
gtgcgtcttgtgagcagaag

SEQ ID NO: 198 - DNA, 20 bp, Artificial Sequence
  Description of Artificial Sequence Synthetic primer
gctacttcaccaacgggagg

SEQ ID NO: 199 - DNA, 20 bp, Artificial Sequence
  Description of Artificial Sequence Synthetic primer
gctacttcaccaacgggagc

SEQ ID NO: 200 - DNA, 22 bp, Artificial Sequence
  Description of Artificial Sequence Synthetic primer
ttcgtgtaccagtttaagggtc

SEQ ID NO: 201 - DNA, 24 bp, Artificial Sequence
  Description of Artificial Sequence Synthetic primer
atttcgtgtaccagtttaagggta

SEQ ID NO: 202 - DNA, 24 bp, Artificial Sequence
  Description of Artificial Sequence Synthetic primer
atttcgtgttccagtttaagggta

SEQ ID NO: 203 - DNA, 24 bp, Artificial Sequence
  Description of Artificial Sequence Synthetic primer
atttcgtgttccagtttaaggcta

SEQ ID NO: 204 - DNA, 21 bp, Artificial Sequence
  Description of Artificial Sequence Synthetic primer
ctcaggaccagagggagggyg

SEQ ID NO: 205 - DNA, 21 bp, Artificial Sequence
  Description of Artificial Sequence Synthetic primer
ctcaggagcagagggagggtg

SEQ ID NO: 206 - DNA, 35 bp, Artificial Sequence
  Description of Artificial Sequence Synthetic primer
gacacaattaagggataaaatctctgaaggagtga

SEQ ID NO: 207 - DNA, 35 bp, Artificial Sequence
  Description of Artificial Sequence Synthetic primer
gacacaattaagggataaaatctctgacggaatga

SEQ ID NO: 208 - DNA, 35 bp, Artificial Sequence
  Description of Artificial Sequence Synthetic primer
gacacaattaagggataaaatctctgaaggaatga

SEQ ID NO: 209 - DNA, 29 bp, Artificial Sequence
  Description of Artificial Sequence Synthetic primer
ggtgcttcccagtaatgagacagggcaca

SEQ ID NO: 210 - DNA, 29 bp, Artificial Sequence
  Description of Artificial Sequence Synthetic primer
ggtgcttcccagtaacgaggcagggcaca

SEQ ID NO: 211 - DNA, 29 bp, Artificial Sequence
  Description of Artificial Sequence Synthetic primer
ggtgcttcccaggaatgagacagggcaca

SEQ ID NO: 212 - DNA, 18 bp, Artificial Sequence
  Description of Artificial Sequence Synthetic primer
ggtgtcctgtccatyctc

SEQ ID NO: 213 - DNA, 12 bp, Artificial Sequence
  Description of Artificial Sequence Synthetic primer
cttgtggsagct

SEQ ID NO: 214 - DNA, 23 bp, Artificial Sequence
  Description of Artificial Sequence Synthetic primer
cgtttcttggagtactctacgtc

SEQ ID NO: 215 - DNA, 23 bp, Artificial Sequence
  Description of Artificial Sequence Synthetic primer
gtttcttggagtactctabgggt

SEQ ID NO: 216 - DNA, 38 bp, Artificial Sequence
  Description of Artificial Sequence Synthetic primer
tgtaaaacgacggccagtattccycgcagaggatttcg

SEQ ID NO: 217 - DNA, 36 bp, Artificial Sequence
  Description of Artificial Sequence Synthetic primer
caggaaacagctatgaccggcgacgccgctcacctc

SEQ ID NO: 218 - DNA, 36 bp, Artificial Sequence
  Description of Artificial Sequence Synthetic primer
caggaaacagctatgaccggcgacgacgctcacctc

SEQ ID NO: 219 - DNA, 36 bp, Artificial Sequence
  Description of Artificial Sequence Synthetic primer
caggaaacagctatgaccgtcaaccacgctcacctc

SEQ ID NO: 220 - DNA, 45 bp, Artificial Sequence
  Description of Artificial Sequence Synthetic primer
cagtcgaggctgatagcgagctcccctgtctgttactgccctcag

SEQ ID NO: 221 - DNA, 45 bp, Artificial Sequence
  Description of Artificial Sequence Synthetic primer
cagtcgaggctgatagcgagctcccctgtctgttactgcccttag

SEQ ID NO: 222 - DNA, 45 bp, Artificial Sequence
  Description of Artificial Sequence Synthetic primer
cagtcgaggctgatagcgagctttcctgtctgttactgcccttag

SEQ ID NO: 223 - DNA, 45 bp, Artificial Sequence
  Description of Artificial Sequence Synthetic primer
cagtcgaggctgatagcgagctctcctgtctgttactgccctcag

SEQ ID NO: 224 - DNA, 44 bp, Artificial Sequence
  Description of Artificial Sequence Synthetic primer
ctatcaacaggttgaactgggcccatagtaacagaaactcaata

SEQ ID NO: 225 - DNA, 44 bp, Artificial Sequence
  Description of Artificial Sequence Synthetic primer
catcgcagtgggctacgtggacgacacgcagttcgtgcggttcg

SEQ ID NO: 226 - DNA, 43 bp, Artificial Sequence
  Description of Artificial Sequence Synthetic primer
tcgcagtgggctacgtggacgacacgcagttcgtgcggttcga

SEQ ID NO: 227 - DNA, 45 bp, Artificial Sequence
  Description of Artificial Sequence Synthetic primer
accggaacacacagatctwcaagrccmasrcacagactgaccgag

SEQ ID NO: 228 - DNA, 44 bp, Artificial Sequence
  Description of Artificial Sequence Synthetic primer
ccggaacacacagatctwcaagrccmasrcacagactgaccgag

(19) United States
(12) Patent Application Publication
(10) Pub. No.: US 2009/0208947 A1
(43) Pub. Date: Aug. 20, 2009

(54) PRIMERS, METHODS AND KITS FOR AMPLIFYING OR DETECTING HUMAN LEUKOCYTE ANTIGEN ALLELES

(75) Inventors: LU WANG, Potomac, MD (US); ROBERT A. LUHM, Wauwatosa, WI (US)

Correspondence Address: LIFE TECHNOLOGIES CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/197,125

(22) Filed: Aug. 22, 2008

Related U.S. Application Data
(63) Continuation of application No. 11/241,871, filed on Sep. 30, 2005, now abandoned.
Application No. 11/241,871 is a continuation-in-part of application No. 10/595,586, filed on Dec. 19, 2006, now abandoned.
(60) Provisional application No. 60/615,326, filed on Oct. 1, 2004.
(60) Provisional application No. 60/515,219, filed on Oct. 28, 2003.
(60) Provisional application No. 60/615,326, filed on Oct. 1, 2004.

(51) Int. Cl. C12Q 1/68; C07H 21/04; C12P 19/34; C40B 30/00

(52) U.S. Cl. 435/6; 536/24.33; 435/91.5; 506/7

(57) ABSTRACT

The present invention describes primers, methods and kits for amplifying and identifying HLA alleles. Using these primers, all HLA alleles at a single locus can be amplified using either a multiplex or non-multiplex PCR approach. Within sets of the primers, control primer pairs may be used to produce control amplicons of a predetermined size from an HLA allele only if a particular HLA locus is present in the sample. The present invention further describes primers for sequencing HLA alleles following amplification. Methods and kits for using the primers are also disclosed.

### PRIORITY CLAIM

The present application specifically claims priority to U.S. Provisional Patent Applications No. 60/615,326, filed Oct. 1, 2004, and to PCT Application No. PCT/04/36044, filed Oct. 28, 2004. The entire disclosure of these priority documents is incorporated by reference herein.

### FIELD OF THE INVENTION

The present invention relates to the amplification, detection and identification of human leukocyte alleles in a sample. More specifically, the present invention relates to methods and materials for the simultaneous amplification of multiple alleles of one or more HLA loci.

### BACKGROUND

A major focus of tissue typing and disease association centers around the human leukocyte antigen (HLA) genes and the alleles encoded by these genes. The human leukocyte antigen complex (also known as the major histocompatibility complex) spans approximately 3.5 million base pairs on the short arm of chromosome 6. The HLA antigen complex is divisible into 3 separate regions which contain the class I, the class II and the class III HLA genes. The HLA genes encompass the most diverse antigenic system in the human genome, encoding literally hundreds of alleles that fall into several distinct subgroups or subfamilies.

Within the class I region exist genes encoding the well characterized class I MHC molecules designated HLA-A, HLA-B and HLA-C. In addition, there are nonclassical class I genes that include HLA-E, HLA-F, HLA-G, HLA-H, HLA-J and HLA-X. HLA A and HLA-C are composed of eight exons and seven introns, whereas HLA-B consists of seven exons and six introns. The sequences of these exons and introns are highly conserved. Allelic variations occur predominantly in exons 2 and 3, which are flanked by noncoding introns 1, 2, and 3. Exons 2 and 3 encode the functional domains of the molecules. The class II molecules are encoded in the HLA-D region. The HLA-D region contains several class II genes and has three main subregions: HLA-DR, -DQ, and -DP.

Recently, researchers have begun using sequence based typing (SBT) to identify the loci and alleles of both class I and class II HLA genes. Unfortunately, the SBT methods currently available in the art do not allow complete resolution of all HLA alleles at a particular loci, such as HLA B because HLA alleles both within and between HLA loci are commonly closely related. Further, the SBT techniques used for allele identification are often time consuming in that they require different reaction conditions and often fail to provide adequate negative and positive controls at initial steps.

In view of the foregoing, what is needed in the art is a convenient and accurate method of determining allelic information from a highly polymorphic system such as the HLA class I and class II regions. Specifically, a need exists to be able to not only resolve all known alleles but identify both class I and class II HLA loci using similar reaction conditions. A further need exists to be able to use the target HLA allele as an amplification reaction control in order to be able to accurately determine the presence of a HLA loci at an initial step of the reaction.

### SUMMARY OF THE INVENTION

In one embodiment a primer set comprising at least two amplification primers capable of amplifying a portion of all human leukocyte antigen alleles of an HLA locus and a control primer pair capable of producing an HLA control amplicon only if the HLA locus is present is described. The control product of HLA origin encompasses a functional aspect of the locus so that additional locus resolution may be obtained.

In other embodiments, a primer set comprising a multiplicity of primers capable of simultaneously amplifying a plurality of a portion of Class I HLA alleles of a HLA locus under a single set of reaction conditions in a multiplex polymerase chain reaction is described. In this embodiment, the primer set may have primers with 5′ non-homologous sequence which may provide all or some of enhanced specificity, more abundant products and more robust reactions, flexibility with respect to primer quality (e.g. tolerance of n−1, n−2, etc., contaminating oligonucleotide primers), and the simultaneous electrophoresis of the sequencing reaction products of multiple loci.

Yet another embodiment discloses a primer for sequencing an HLA allele that comprises a 3′ portion that is complementary to an HLA allele and a 5′ portion that is not complementary to an HLA allele, wherein the primer allows complete resolution of an exonic sequence of the HLA allele during a sequencing reaction. In these embodiments, the 5′ non-homologous sequence may provide all or some of enhanced specificity, more abundant products and more robust reactions, flexibility with respect to primer quality, and the simultaneous electrophoresis of the sequencing reaction products of multiple loci.

Based on these primers and primer sets, methods of amplifying and detecting HLA alleles using the primers and primer sets are described. Kits for carrying out these methods are also provided in some embodiments. These kits can include instructions for carrying out the methods, one or more reagents useful in carrying out these methods, and one or more primer sets capable of amplifying all HLA alleles.

Objects and advantages of the present invention will become more readily apparent from the following detailed description.

### BRIEF DESCRIPTION OF THE DRAWINGS

FIGS. 1A and 1B show agarose gels illustrating amplification results obtained using the primers and primer set of the present invention. FIGS. 1A and 1B exhibit positive amplification of HLA A locus alleles and HLA B locus alleles, respectively.

FIGS. 2A-2D show sequencing electropherograms from the alleles amplified and sequenced in the examples.

FIG. 3 shows an agarose gel illustrating DRBI amplification results on five different samples obtained using the primers and primer sets of the present invention.

### DETAILED DESCRIPTION

The present invention relates to primers, primer pairs and primer sets for amplifying and/or sequencing HLA alleles and to methods for amplifying and detecting HLA alleles. In some embodiments, the methods of detecting comprise sequencing methods. The invention is based, at least in part, on the inventors' identification of novel primer sequences for amplifying and/or sequencing HLA alleles. Generally, the primers provided herein may be used to amplify any HLA alleles present in a sample. Accordingly, the primers and methods may be used for research and clinical applications for any HLA associated disease, disorder, condition or phenomenon.

The primers, primer pairs, primer sets, and methods of the present invention not only strengthen amplification and sequencing reaction robustness, but they also provide specificity and product stability not seen with other primers or methods of HLA sequence-based typing. Moreover, the primers, primer sets and methods of the present invention allow similar amplification and cycle sequencing times such that unrelated target sequences can be processed en masse. Electrophoresis times for sequencing of the amplification product is also standardized so that these processes can be performed concurrently regardless of the sequence or size of the initial DNA template.

Some of the primer pairs and primer sets are designed for use in multiplex amplifications wherein multiple alleles from one or more HLA loci are amplified simultaneously under the same, or substantially similar, reaction conditions. Amplification methods that use control primer pairs are also provided. The use of these control primer pairs is advantageous because it allows the user to determine whether an HLA allele amplification was successful and to identify false positives within the amplification data.

The primers and methods provided herein may be used in the amplification of any known HLA alleles of any HLA locus. Moreover, the methods may even be extended to as yet unknown HLA alleles. For example, HLA loci that may be used as target sequences in the amplifications include, but are not limited to, the HLA-A locus, the HLA-B locus, the HLA-C locus, the HLA-D locus (including HLA-DP, HLA-DQ and HLA-DR), the HLA-E locus, the HLA-F locus, the HLA-G locus, the HLA-H locus, the HLA-J locus and the HLA-X locus. In some instances the present methods may be directed to multiplex amplifications that use one or more (e.g., all) loci of a given class of HLA loci as target sequences. HLA loci classes are well known. These include Class I and Class II loci. Class I encompasses the following alleles: alleles of the HLA-A, -B, -C, -E, -F, and -G loci. Class II encompasses the following alleles: HLA-DRA, HLA-DRB1, HLA-DRB2-9, HLA-DQA1, HLA-DQB1, HLA-DPA1, HLA-DPB1, HLA-DMA, HLA-DMB, HLA-DOA and HLA-DOB.

One aspect of the invention provides novel primer sequences for amplifying and/or sequencing HLA alleles. Table 1 presents a list of primers that may be used to amplify HLA alleles in accordance with the present invention. The list includes the sequence of each primer, as well as the HLA loci which the primer is capable of amplifying. As noted in the table, the primers include amplification and sequencing primers for single product reactions (i.e. primers used to amplify multiple HLA alleles at a specific loci using a single full length product where some reactions include the amplification of a control), multiplex product reactions for different HLA loci (i.e., primers used to amplify multiple HLA alleles at a specific loci using multiple smaller products where some reactions include the amplification of a control), group specific single tube and multitube multiplex primers (i.e. primers used in amplifying and sequencing alleles at more than one loci using a single full length product where some reactions include the amplification of a control), and potential group sequencing primers. The group specific sequencing primers are primers that will anneal to specific allelic groups based upon a common motif in the target sequence. It should be understood that classifying a primer as a group sequencing primer is not entirely restrictive as known allele assignments do not necessarily reflect the sequence at the hypervariable region. As demonstrated in Table 1, the group specific sequencing primers yGSDR-07, 04, 02, 01, 03/5/6, 07, and 08/12 are examples of group specific sequencing primers that anneal to a common motif found in DRB 1. The codon 86 primers are examples of group specific sequencing primers that recognize the specific dual motif at codon 86 in DRB 1. Potential group sequencing primers include primers that should anneal based on common motifs. Thus, the potential group specific sequencing primers yDQ2, 3, 4, 5, 6A, 6TA, and 6TCA of DQB 1 were designed using a common motif specific for DQB 1. Although Table 1 does not disclose potential group specific sequencing alleles for all loci, the design of these primers based on loci specific common motifs can be extended to all HLA loci.

The sequence of each primer oligonucleotide is selected such that it is complementary to a predetermined sequence of the target molecule. The primer oligonucleotides typically have a length of greater than 10 nucleotides, and more preferably, a length of about 12-50 nucleotides, such as 12-25 or 15-20. However, in some embodiments, the 3′ terminus of the primers of the primer sets are capable of being extended by a nucleic acid polymerase under appropriate conditions and can be of any length, for example ranging from about 5 nucleotides to several hundred. In any case, the length of the primer should be sufficient to permit the primer oligonucleotides to hybridize to the target molecule. In some embodiments, the primer oligonucleotides can be chosen to have a desired melting temperature, such as about 40 to about 80° C., about 50 to about 70° C., about 55 to about 65° C., or about 60° C.

In certain embodiments, the amplification primers will have a 5′ portion containing a non-homologous sequence that does not hybridize to the HLA allele, but can provide enhanced specificity of amplification of the target sequence. In Table 1, amplification primer sequence non-homologous to the HLA sequence are demonstrated by being listed in italics. As a non-limiting theory, it is believed that this increased specificity results from the lowering of the strength of binding (Tm) to more than one HLA locus as compared to a completely homologous primer by providing a primer with initial weaker binding. However, a more abundant product and more robust amplification as compared to using a completely homologous primer is still obtained because once the amplification reaction begins, the non-homologous sequences are incorporated into the product, thus providing homologous sequences when subsequent primers bind during further amplification. The addition of 5′ non-homologous sequences to the amplification primers also provides some flexibility with respect to primer quality as the amplification reactions tend to be more tolerant to contamination with other primers. It also saves time and reaction components by allowing a single run of electrophoresis of all loci amplification products. As one of skill in the art understands, with some primers only some of these advantages may be evident. Other primers demonstrating non-homologous sequence may encompass all of the advantages set forth above.

Although the present primers generally utilize the five standard nucleotides (A, C, G, T and U) in the nucleotide sequences, the identity of the nucleotides or nucleic acids used in the present invention are not so limited. Non-standard nucleotides and nucleotide analogs, such as peptide nucleic acids and locked nucleic acids can be used in the present invention, as desired. In the reported sequences, letters other than A, C, G or T indicate non-standard universal bases as follows: R, Y, S, M, W, and K are degenerate bases consisting of two possible bases at the same position. A or G=R, C or T=Y, G or C=, C or A=M, A or T=W and G or T=K. There are also combinations of 3 possible bases at a particular base position known as H, B, V.

Nucleotide analogs are known in the art (e.g., see, Rawls, C & E News Jun. 2, 1997: 35; Brown, Molecular Biology LabFax, BIOS Scientific Publishers Limited; Information Press Ltd, Oxford, UK, 1991). When used with the primers, primer sets and methods of the present invention, these nucleotide analogs may include any of the known base analogs of DNA and RNA such as, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, hypoxanthine, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, orotic acid, 2,6-diaminopurine and the AEGIS™ bases isoC and isoG. As such, the primers can contain DNA, RNA, analogs thereof or mixtures (chimeras) of these components. In addition to the use of non-standard nucleotides and nucleotide analogs, the bases in the primer sequences may be joined by a linkage other than a phosphodiester bond, such as the linkage bond in a peptide nucleic acid, as long as the bond does not interfere with hybridization.

Universal nucleotides can also be used in the present primers. In some instances, nucleotide analogs and universal nucleotides will encompass the same molecules. As used herein, universal nucleotide, base, nucleoside or the like, refers to a molecule that can bind to two or more, i.e., 3, 4, or all 5, naturally occurring bases in a relatively indiscriminate or non-preferential manner. In some embodiments, the universal base can bind to all of the naturally occurring bases in this manner, such as 2′-deoxyinosine (inosine). The universal base can also bind all of the naturally occurring bases with equal affinity, such as 3-nitropyrrole2′-deoxynucleoside (3-nitropyrrole) and those disclosed in U.S. Pat. Nos. 5,438,131 and 5,681,947. Generally, when the base is “universal” for only a subset of the natural bases, that subset will generally either be purines (adenine or guanine) or pyrimidines (cytosine, thymine or uracil). An example of a nucleotide that can be considered universal for purines is known as the “K” base (N6-methoxy-2,6-diaminopurine), as discussed in Bergstrom et al., Nucleic Acids Res. 25:1935 (1997). And an example of a nucleotide that can be considered universal for pyrimidines is known as the “P” base (6H,8H-3,4-dihydropyrimido[4,5-c][1,2]oxazin-7-one), as discussed in Bergstrom et al., supra, and U.S. Pat. No. 6,313,286. Other suitable universal nucleotides include 5-nitroindole (5-nitroindole 2′-deoxynucleoside), 4-nitroindole (4-nitroindole 2′-deoxynucleoside), 6-nitroindole (6-nitroindole 2′-deoxynucleoside) or 2′-deoxynebularine. When universal nucleotides are used, a partial order of base-pairing duplex stability has been found as follows: 5-nitroindole >4-nitroindole >6-nitroindole >3-nitropyrrole. When used, such universal bases can be placed in one or more polymorphic positions, for example those that are not required to specifically identify an allele. Combinations of these universal bases at one or more points in the primers can also be used as desired. Primers and strategies using universal primers are discussed in U.S. patent application Ser. No. 10/429,912.

In some embodiments, deazaG is used in order to increase the amplification of certain alleles that when in combination with other alleles will not amplify when all “natural” nucleotide primers are used. The addition of deazaG increases amplification of loci with high GC percentages, such as what is found in many of the class I loci.

The primers of Table 1 may be used as primer pairs and primers sets in a variety of combinations. Although primer pairs are often used in nucleic acid amplifications, the present primer sets can contain odd numbers of primers so that one or more forward primers can work in conjunction with a single reverse primer to produce an amplicon and vice versa. It is to be understood that any combination of the primers listed in Table 1 can be combined into a primer set. The only requirement is that the assembled primer set be capable of performing at least one step in one or more of the methods of the present invention. The primer sets in Table 1 labeled group specific or multiplex primers give examples of primer sets that have been assembled. Each individual section of Table 1 demonstrates embodiments of primer sets of the present invention. The skilled artisan will understand that individual primers or combinations of primers that encompass less than the entire section of Table 1 may be used in alternative embodiments.

The locations of hybridization for the primer pairs is desirably designed to provide amplicons that span enough polymeric positions of a locus to allow for individual alleles of the locus to be resolved in a subsequent sequencing reaction. This will generally be referred to as spanning a “portion” of a HLA allele. In some embodiments, the primers shown in Table 1 can be varied by one, two, five, ten, twenty or more positions on the HLA allele, or any number of positions between one and twenty, either upstream or downstream, and still provide acceptable results. As used herein, acceptable results generally encompass results where there will be resolution of the functional aspect of the HLA locus with sequence of sufficient quality to provide unambiguous HLA typing for that locus. The skilled artisan will understand that unambiguous HLA typing as an acceptable result does not mean the complete elimination of ambiguities, rather it means that the data generated is unambiguous. Typically, in embodiments where the primer hybridization position is moved upstream of the position illustrated in Table 1, additional bases that hybridize to the HLA allele further upstream of the primer demonstrated in Table 1 will be added. Similarly, when the hybridization position is moved downstream, then bases are added to the primer that hybridize to the HLA allele downstream. In many embodiments, when the hybridization position of the primer demonstrated in Table 1 is moved either upstream or downstream, this will be accompanied by removal of bases from the end of the primer opposite the end moved either upstream or downstream.

The primers of the present invention are well-suited for use in the amplification of HLA alleles. Amplification using the primers may be carried out using a variety of amplification techniques, many of which are well-known. Suitable amplification techniques include those which use linear or exponential amplification reactions. Such techniques include, but are not limited to, polymerase chain reaction (PCR), transcription based amplification and strand displacement amplification. For example, the primers are readily applicable to RT PCR of HLA mRNA for expression analysis because they target exon regions. During amplification, the type of nucleic acid (e.g., RNA, DNA and/or cDNA) amplified by the primers and primers sets is not particularly limiting as long as the primers can hybridize and amplify the target nucleic acid in the sample. One of skill in the art will understand that if cDNA is amplified during an amplification reaction, cDNA will be sequenced during the subsequent sequencing reaction. In some embodiments, RT-PCR will be used to reverse transcribe RNA and amplify the cDNA that results. This method is well-known in the art and several commercial kits exist. One of skill in the art will understand that in some embodiments RNA will be the preferred starting material.

The skilled artisan will understand that the sample from which the nucleic acid to be amplified derives can encompass blood, bone marrow, spot cards, RNA stabilization tubes, forensic samples, or any other biological sample in which HLA alleles can be amplified. Generally, the sample to be detected can be obtained from any suitable source or technique. The nucleic acid may also be isolated from the sample using any technique known in the art. In some embodiments, the sample will be genomic DNA. In many embodiments, the nucleic acid will not be isolated from the sample before the amplification reaction. In other embodiments, the nucleic acid will be isolated from the sample prior to amplification.

The primer pairs and sets may be used in both non-multiplex and multiplex amplifications. For example, a non-multiplex amplification may be used to amplify some or all of the alleles of a single locus, while a multiplex amplification may be used to amplify simultaneously alleles of different loci.

As one of skill in the art would recognize, multiplex amplifications may offer significant advantages over non-multiplex amplifications in terms of time and efficiency. Recognizing this, another aspect of the invention provides methods for multiplex amplification of human leukocyte antigen (HLA) alleles based on the use of primer pairs or primer sets capable of simultaneously amplifying multiple alleles from one or more HLA loci.

Generally, primer pairs and sets may be selected to amplify any HLA alleles present in a genomic sample using a multiplex amplification approach. The selection of an appropriate primer pair or primer set for a particular multiplex amplification will depend on the alleles and loci that are to be amplified. An appropriate primer pair or primer set should be selected such that it is capable of amplifying multiple alleles from the selected locus or loci under the same (or very similar) amplification conditions and protocols. Many different combinations of primers from Table 1 may be suitable for use in the present multiplex applications. Several examples of such combinations are provided in the Examples section below. In some embodiments, the primers used in multiplex reactions will have 5′ portions with non-homologous sequence.

In some embodiments of the present invention, a multiplex amplification is used to amplify a plurality of portions of a single HLA locus. Generally, where a plurality of portions of a single HLA allele are to be amplified, the primer pairs or sets desirably include a multiplicity of primers that hybridize to multiple non-allele specific regions of the HLA loci. This hybridization to non-allele specific regions allows all different HLA alleles to be successfully amplified. In many cases, following multiplex amplication using the multiplicity of primers, the plurality of amplicons produced will cover some overlapping sequence.

In other embodiments of the present invention, multiplex amplification is used to amplify multiple HLA alleles from two or more HLA loci. This includes embodiments where a multiplex amplification is used to amplify all HLA alleles of two or more HLA loci. Although each HLA locus is physically distinct, with some being separated by large distances, in some embodiments all loci may be amplified in a single multiplex reaction which amplifies all or a selected subgroup of clinically significant loci. For example, in some illustrative embodiments all alleles of the two or more HLA loci may be amplified simultaneously in a single vessel by using an appropriate primer set, as provided herein. Where alleles from more than one loci are to be amplified, the primer set desirably includes a primer pair that is specific to each locus to be amplified. In some embodiments, the multiplex amplification of alleles from different HLA loci is achieved while maintaining individual locus specificity because the product sizes produced from the amplification of individual loci differ in size and, therefore, may be separated by, for example, electrophoresis or chromatography.

Different amplification strategies may be employed for amplifying the alleles of different HLA loci. For example, a non-multiplex amplification approach may be sufficient for the amplification of alleles that are relatively easily resolved. Thus, where alleles of the HLA A locus are being amplified, a non-multiplex amplification may be employed where primers are selected to provide a single amplicon that includes exons 2, 3 and 4. In still other embodiments, the present methods may be used to amplify multiple, and, in some cases, all, alleles of a particular class of HLA loci. For example, the present methods may be employed to amplify multiple (e.g., all) alleles of the Class I HLA loci. Similarly, the present methods may be employed to amplify multiple (e.g., all) alleles of the Class II HLA loci. An amplification of this type is described in detail in Example 1, below.

On the other hand, a multiplex amplification may be more desirable when the alleles of a given locus are difficult to resolve. Such may be the case for HLA alleles of the HLA B locus and HLA alleles for the HLA DR locus. Thus, where HLA B locus alleles are being amplified, different primer pairs within a primer set can be used simultaneously to produce dual amplicons that cover exons 2, 3 and 4. The use of two primer pairs in a single amplification of the B locus has the advantage of reducing the number of potential heterozygotic combinations. This results in simplified sequence analysis and a further reduction of the number of resultant ambiguities. These advantages can be achieved, for example, by simultaneously amplifying as two or more distinct groups the regions from exon 1 to intron 3 and intron 3 to exon 5 as two separate products in one amplification mix. This results in a much more robust amplification than the non-multiplex amplification of a single product. Additionally, amplifying the HLA B locus as two separate products is advantageous over a single product amplification as a single product is frequently weak, making it difficult to discern using detection methods such as agarose electrophoresis. This difficulty is particularly prominent when modified nucleotides are required. One of skill in the art will understand that when using a multiplicity of primers in multiplex amplification, certain primers in each primer pair can be common. For example, in a multiplex amplification, two (or more) forward primers may be used with a single reverse primer. There is no requirement that an equal number of individual forward and reverse primers be used in each multiplex amplification.

Multiplex amplification is also desirably used in the amplification of alleles of the HLA DR locus. For this reason, one embodiment of the invention provides a multiplex amplification of alleles of the HLA DR locus using a primer set that allows for eleven group specific amplifications that achieve resolution of alleles DRB1, DRB3, DRB4, and DRB5 within exon 2. Although in certain embodiments, this multiplex amplification will consist of amplification of only a single product plus the HLA control, these reactions can be amplified simultaneously as they require similar or identical reaction conditions. An amplification of this type is described in detail in Example 1, below. Although the primer sets are envisioned to resolve regions outside of DR locus exon 2, resolving exon 2 currently has special significance as the standard convention in the transplant community is that only resolution of exon 2 is relevant for DR tissue matching. The skilled artisan will understand that this may likely change with time, as several ambiguities remain unresolved by only using an exon 2 resolution approach.

Another aspect of the invention provides for the use of control primer pairs in HLA allele amplifications. These control primer pairs may be included in the amplifications (non-multiplex and multiplex) in order to verify the success and accuracy of the amplification. The amplicon produced by amplification using these control primer pairs may also be used to specifically identify certain alleles, i.e. the amplicon produced by the control primer pair may be sequenced. Generally, these control primers operate by producing a control amplicon (i.e., a product produced from the amplification of an HLA allele) whenever one or more HLA alleles are present within a sample. Using control primers that amplify an HLA allele is advantageous as they provide a mechanism to ensure that DNA has in fact been added to the amplification reaction. In addition, the control primers may provide an indication of the efficiency of any HLA allele amplification and may identify false positive results. For example, if the results of the amplification provide an amplicon but lack the control amplicon, then the amplicon is likely a false positive. In contrast, if the control amplicon is also present, then the amplification produced a positive result.

In some embodiments, the control primers amplify a ubiquitous gene in a sample. In these embodiments, primers to any gene that can serve as an adequate reaction control may be used. Non-limiting examples include primers that amplify the GAPDH housekeeping genes. In preferred embodiments, however, the control primers use target HLA alleles as templates. In order to provide an effective control, the portion of the HLA allele amplified by the control primer pair is desirably common to all or substantially similar to all HLA alleles being tested. Thus, a control amplicon will be produced if any of the alleles of interest are present. When multiple HLA loci are being amplified with the primer sets of the present invention, a control primer pair common to all or substantially all of the HLA alleles at a particular loci is desirably included for each loci. As long as the control primer pair does not interfere with the primary amplification, the control primer pair can span a region with or without polymorphic positions. Accordingly, the portion of the HLA allele amplified by the control primer pair can have base polymorphisms as well as insertions or deletions. As used herein, a portion of an HLA allele is substantially similar when the control primers are capable of binding to the allele and producing an amplicon.

In additional embodiments, particularly when the target HLA locus is HLA A, HLA B, or HLA C the portion of the HLA allele amplified by the control primer pair comprises all of exon 4 and beyond exon 4. In other embodiments, the control primer pair amplifies all of exon 4 and all of exon 5 of the HLA allele. In yet further embodiments, the control primer pair amplifies all of exon 4, exon 5, exon 6, exon 7, and exon 8. In these embodiments, the primer set can be used in an amplification reaction to amplify an HLA allele and also provide a control. Thus, the presence or absence of a control amplicon in an amplification reaction may be used to confirm the presence or absence HLA alleles in a sample.

The molecular weight of the control amplicon is desirably predetermined, meaning that the expected size of the product from the control reaction will be known prior to the reaction. This allows the user to quickly check for the HLA control amplicon using electrophoresis (e.g., gel electrophoresis), in order to determine the success of the amplification reaction. The size of the control amplicon is not particularly limiting and can be any size capable of amplification and detection, including but not limited to less than 500, 500-600, 600-700, 700-800, 800-900, 900-1000, or more than 1000 or 2000 base pairs in length.

Following the amplification of the HLA alleles in a sample, the alleles may be detected and/or sequenced. Thus, another aspect of the invention provides methods and assays for the detection of specific alleles in a sample. Optionally, the amplicons may be treated to remove unused primers prior to the detection of amplification products.

In one basic embodiment of a detection assay provided by the present invention, a sample containing, or suspected of containing, an HLA allele or HLA locus will be contacted with primer pairs or sets, as provided herein, under conditions in which individual primer pairs will amplify the HLA allele or locus for which the primer pair or set is specific. The production of an amplicon will indicate the presence of an HLA allele or locus in a sample. In many embodiments, the presence or absence of an amplicon will be compared to the presence or absence of a control amplicon.

The presence or absence of an amplicon may be determined by standard separation techniques including electrophoresis, chromatography (including HPLC and denaturing-HPLC), or the like. Primer labels may be used in some detection schemes. In these schemes the primers are labeled with a detectable moiety. Suitable examples of detectable labels include fluorescent molecules, beads, polymeric beads, fluorescent polymeric beads and molecular weight markers. Polymeric beads can be made of any suitable polymer including latex or polystyrene. One of skill in the art understands that any detectable label known in the art may be used with the primers and primer sets as long as the detectable label does not interfere with the primers, primer sets or methods of the invention.

Detection of alleles in a sample may also be carried out using a primer array. In such an array primer pairs and/or primer sets, as provided herein, are contained within distinct, defined locations on a support. The skilled artisan understands that arrays can be used with the amplification and/or sequencing primers, primer sets and methods of the present invention. Any suitable support can be used for the present arrays, such as glass or plastic, either of which can be treated or untreated to help bind, or prevent adhesion of, the primer. In some embodiments, the support will be a multi-well plate so that the primers need not be bound to the support and can be free in solution. Such arrays can be used for automated or high volume assays for target nucleic acid sequences.

In some embodiments, the primers will be attached to the support in a defined location. The primers can also be contained within a well of the support. Each defined, distinct area of the array will typically have a plurality of the same primers. As used herein the term “well” is used solely for convenience and is not intended to be limiting. For example, a well can include any structure that serves to hold the nucleic acid primers in the defined, distinct area on the solid support. Non-limiting example of wells include depressions, grooves, walled surroundings and the like. In some of the arrays, primers at different locations can have the same probing regions or consist of the same molecule. This embodiment is useful when testing whether nucleic acids from a variety of sources contain the same target sequences. In many embodiments, the solid support will comprise beads known in the art. The arrays can also have primers having one or multiple different primer regions at different locations within the array. In these arrays, individual primers can recognize different alleles with different sequence combinations from the same positions, such as, for example, with different haplotypes. This embodiment can be useful where nucleic acids from a single source are assayed for a variety of target sequences. In certain embodiments, combinations of these array configurations are provided such as where some of the primers in the defined locations contain the same primer regions and other defined locations contain primers with primer regions that are specific for individual targets.

Yet another aspect of the invention provides primers for sequencing the HLA alleles contained in the amplicons obtained using the present amplification methods. The sequencing reactions use primer pairs and primer sets that are separate and distinct from the primer pairs and sets used in the amplification of the alleles. However, similarly to the amplification primers, the sequencing primers may be used in multiplex reactions. The combination of HLA allele amplification followed by sequencing in accordance with the present invention allows the resolution of many of the HLA alleles. Accordingly, in some embodiments, the amplification and sequencing primer pairs and sets can be used to resolve greater than or about 50%, 55%, 60%, 65%, 70%, 75%, 80% or more of cis/trans ambiguities, including those found in the HLA B locus. Certain embodiments for resolving cis/trans ambiguities on the HLA B locus will encompass two separate multiplex amplification reactions.

The sequencing primers may be used in a variety of sequencing protocols, many of which are well-known. One such protocol is the Sanger sequencing protocol. This sequencing protocol can be facilitated using DYEnamic™ ET* Terminator Cycle Sequencing Kits available from Amersham Biosciences (Piscataway; N.J.). Other suitable sequencing protocols include sequencing by synthesis protocols, such as those described in U.S. Pat. Nos. 4,863,849, 5,405,746, 6,210,891, and 6,258,568; and PCT Applications Nos. WO 98/13523, WO 98/28440, WO 00/43540, WO 01/42496, WO 02/20836 and WO 02/20837, the entire disclosures of which are incorporated herein by reference.

Examples of suitable sequencing primers for use in the present sequencing methods are provided in Table 1, including SEQ. ID. Nos. 14-21, 53-77, 103-119, 131-132, 148-164, 185-186, and 197-203. When using the sequencing primers of Table 1, complete exon sequences can be determined in some instances. In many embodiments, multiple sequencing primers will be used in individual reactions to produce a multiplex sequencing reaction. Multiplex sequencing reactions have many of the same advantages as multiplex amplification reactions. In some embodiments, the multiplex sequencing reaction will comprise whole locus sequencing of various HLA loci. In other embodiments, the multiplex sequencing reaction will comprise partial loci sequencing of various HLA loci.

In some of the sequencing primers, the 5′ portion of the sequencing primer contains a non-homologous sequence that does not hybridize to the HLA allele but can provide enhanced resolution of the sequence generated early in the polymerization reaction. In Table 1, sequencing primer sequence non-homologous to the HLA sequence are demonstrated by being listed in italics. By having or adding additional non-homologous bases to the 5′ end of the sequencing primer, the non-complementary portion can achieve enhanced resolution of sequence. Without wishing or intending to be bound to any particular theory of the invention, the inventors believe that this increased resolution occurs because the first bases resolved on any sequencing system are unclear. Clarity tends to improve within 30 to 35 bases from the 5′ end of the sequencing primer as the time in the capillary of the sequencer is increased. Thus, a primer design encompassing additional non-homologous bases is particularly useful in sequencing primers that hybridize close to, for example within 10, 15, 20, 25, 30 or bases, of an intron/exon junction, such as where locus structure dictates placement of the primer close to the junction, such as that required with exons 2 and 3. Generally, the number of the additional non-hybridizing bases added to the 5′ end of the sequencing primers can vary as desired. For example one to 35 bases (e.g., 2, three, four, five, ten, fifteen, or twenty bases) may be added to the 5′ end. 5′ modification also results in increased specificity as the strength of binding of the sequencing primer is lower as compared to a completely homologous primer. For these reasons, a stronger and more robust sequencing reaction as compared to using a sequencing primer without 5′ amplification is obtained. The addition of bases to the sequencing primer also insure that all sequencing products are approximately the same size and can be read in-frame. Having sequencing products of the same size saves time and reaction components by allowing a single electrophoretic run of all loci sequencing products because they all fall within the same range of links.

Sequencing primer designs that use additional non-homologous bases are also advantageous because many transplant clinics demand that the exons, such as exon 3, be covered completely with usable sequence. Where the exon sequence is very close to the 3′ end of a sequencing primer, the sequence tends to be poorly resolved and valuable exonic data is lost during sequencing. In light of this, in certain embodiments of the invention, it is advantageous to place the sequencing primer far enough away from the intron/exon junction so that this near resolution is not an issue. Unfortunately, with some HLA loci, especially the class I loci, there are commonly insertion/deletion events near the intron/exon junctions. In some of these loci, depending on the allelic combination, sequencing primers cannot be placed upstream to an insertion/deletion because of resulting unreadable sequence. In these cases, it is preferential to anneal the primers near the junctions. In these cases, when the primers are near the intron/exon junctions, the addition of non-homologous bases to the primers provides additional sequence clarity.

In some embodiments, a multiplex sequencing approach will be partially based on fluorescently labeled locus specific sequencing primers. When primers containing specific fluorescent labels with specific emission wavelengths assigned to specific loci are used in a multiplex sequencing reaction, the combination of the 5′ non-homologous sequence with the fluorescent signature could discriminate the allele generated at each loci even when multiple sequencing reaction are occurring in a single tube.

Following sequencing, the sequencing product may be treated to remove excess terminators, resuspended and denatured and resolved on a sequencer to obtain a final allele assignment.

A final aspect of the invention provides kits for carrying out the methods described herein. In one embodiment, the kit is made up of one or more of the described primers or primer sets with instructions for carrying out any of the methods described herein. The instructions can be provided in any intelligible form through a tangible medium, such as printed on paper, computer readable media, or the like. A plurality of each primer or primer set can be provided in a separate container for easy aliquoting. The present kits can also include one or more reagents, buffers, hybridization media, salts, nucleic acids, controls, nucleotides, labels, molecular weight markers, enzymes, solid supports, dyes, chromatography reagents and equipment and/or disposable lab equipment, such as multi-well plates (including 96 and 384 well plates), in order to readily facilitate implementation of the present methods. Such additional components can be packaged together or separately as desired. One of skill in the art will understand that both the amplification and the sequencing methods of the present invention lend to being carried out on solid supports. Solid supports can include beads and the like whereas molecular weight markers can include conjugatable markers, for example biotin and streptavidin or the like. Enzymes that can be included in the present kits include DNA polymerases and the like. In some embodiments, kits include all reagents, primers, equipment etc. needed to perform the HLA amplification and/or sequencing except for the sample to be tested. Examples of kit components can be found in the description above and in the following examples. In some embodiments, the kits of the invention will include all of primers in Table 1 that are in bold lettering. One of skill in the art will understand that the primers in bold in Table 1 may be used together to accomplish many of the methods of the invention.

TABLE 1
* All primers in Table 1 are written in the 5′ to 3′ direction

| | | | | | | |
|---|---|---|---|---|---|---|
| | | | | | Amount/ | Final |
| Primer ID | Locus | Primer Type | Primer Sequence | Location | rxn | Molarity |

A Locus Single Product Primers

| | | | | | | |
|---|---|---|---|---|---|---|
| pA5-3 | HLA-A | amp primer | CAGACSCCGAGGATGGCC | * 20,766,431- | 0.5 μl | 20 μM |
| | | | (SEQ ID NO.: 1) | 20,766,448 | | |
| pA3-29 | HLA-A | amp primer | GCAGCGACCACAGCTCCAG | * 20,768,461- | 0.5 μl | 20 μM |
| | | | (SEQ ID NO.: 2) | 20,768,479 | | |
| pA5-5 | HLA-A | 5′ amp primer | ACCAGAAGTCGCTGTTCCCTYYTCAGGGA | * 20,767,819- | 0.5 μl | 20 μM |
| | | | (SEQ ID NO.: 3) | 20,767,847 | | |
| pA3-31 | HLA-A | 3′ amp primer | AAAGTCACGGKCCCAAGGCTGCTGCCKGTG | * 20,767,697- | 0.5 μl | 20 μM |
| | | | (SEQ ID NO.: 4) | 20,767,726 | | |
| pA3-29-2 | HLA-A | amp primer | TCACRGCAGCGACCACAGCTCCAG | * 20,768,456- | 0.5 μl | 20 μM |
| | | | (SEQ ID NO.: 5) | 20,768,479 | | |
| pA3x23b | HLA-A | 3′ amp primer | CTC AGG ACC AGA GGG AGG GYG | * 20,767,528- | 1.0 μl | 10 μM |
| | | | (SEQ ID No.:204) | 20,767,550 | | |
| pA3x23b80 | HLA-A | 3′ amp primer | CTC AGG AGC AGA GGG AGG GTG | * 20,767,528- | 1.0 μl | 10 μM |
| | | | (SEQ ID NO.:205) | 20,767,550 | | |
| A 3′ UT | HLA-A | amp primer | GCCTTTGCAGAAACAAAGTCAGGGTTC | * 20,769,409- | 0.5 μl | 20 μM |
| | | | (SEQ ID NO.: 6) | 20,769,435 | | |
| pA5-3 + 3 | HLA-A | 5′ amp primer | CCCCAGACSCCGAGGATGGCC | * 20,766,428- | 0.5 μl | 20 μM |
| | | | (SEQ ID NO.: 7) | 20,766,648 | | |
| pA3-31 + 3 | HLA-A | 3′ amp primer | GGAAAAGTCACGGKCCCAAGGCTGCTGCCKGTG | * 20,767,695- | 0.5 μl | 20 μM |
| | | | (SEQ ID NO.: 8) | 20,767,726 | | |
| pA5-9a + 3 | HLA-A | 5′ amp primer | CTTGTTCTCTGCTTCCCACTCAATGTGTG | * 20,767,738- | 0.5 μl | 20 μM |
| | | | (SEQ ID NO.: 9) | 20,767,766 | | |
| pA5-x4a1 | HLA-A | 5′ amp primer | GAC ACA ATT AAG GGA TAA AAT CTC | * 20,767,620- | 1.0 μl | 10 μM |
| | | | TGA AGG AGT GA | 20,767,654 | | |
| | | | (SEQ ID No.: 206) | | | |
| pA5-x4a2 | HLA-A | 5′ amp primer | GAC ACA ATT AAG GGA TAA AAT CTC | * 20,767,620- | 1.0 μl | 10 μM |
| | | | TGA GGG AAT GA | 20,767,654 | | |
| | | | (SEQ ID No.: 207) | | | |
| pA5-x4a3 | HLA-A | 5′ amp primer | GAC ACA ATT AAG GGA TAA AAT CTC | * 20,767,620- | 1.0 μl | 10 μM |
| | | | TGA AGG AAT GA | 20,767,654 | | |
| | | | (SEQ ID No.: 208) | | | |
| pA3-39 + 3 | HLA-A | Ex4 amp primer | GCTGAGATCAGGTCCCATCACTGCCGTA | * 20,768,704- | 0.5 μl | 20 μM |
| | | | (SEQ ID NO.: 10) | 20,768,731 | | |
| pA3-40 + 4 | HLA-A | Ex4 amp primer | GCTGAGATCAGGTCCCATCAGCGCTGTA | * 20,768,704- | 0.5 μl | 20 μM |
| | | | (SEQ ID NO.: 11) | 20,768,731 | | |
| pA3-42 + 3 | HLA-A | Ex4 amp primer | GCTGAGATCAGGTCCCATCACCGCCATA | * 20,768,704- | 0.5 μl | 20 μM |
| | | | (SEQ ID NO.: 12) | 20,768,731 | | |
| pA3-43 + 3 | HLA-A | Ex4 amp primer | GCTGAGATCAGGTCCCATCACCGCCGTA | * 20,768,704- | 0.5 μl | 20 μM |
| | | | (SEQ ID NO.: 13) | 20,768,731 | | |
| pA3-x4b1 | HLA-A | Ex4 amp primer | GGT GCT TCC CAG TAA TGA GAC AGG | * 20,768,171- | 1.0 μl | 10 μM |
| | | | GCA CA | 20,768,199 | | |
| | | | (SEQ ID No.: 209) | | | |
| pA3-x4b2 | HLA-A | Ex4 amp primer | GGT GCT TCC CAG TAA CGA GGC AGG | * 20,768,171- | 1.0 μl | 10 μM |
| | | | GCA CA | 20,768,199 | | |
| | | | (SEQ ID No.: 210) | | | |
| pA3-x4b3 | HLA-A | Ex4 amp primer | GGT GCT TCC CAG GAA TGA GAC AGG | * 20,768,171- | 1.0 μl | 10 μM |
| | | | GCA CA | 20,768,199 | | |
| | | | (SEQ ID No.: 211) | | | |
| Aex2F | HLA-A | seq primer | GGGAAAGSGCCTCTG | * 20,766,534- | 0.5 μl | 20 μM |
| | | | (SEQ ID NO.: 14) | 20,766,548 | | |
| Aex2R-4 | HLA-A | seq primer | GGATCTCGGACCCGGAGACTGT | * 20,766,982- | 1 μl | 3 μM |
| | | | (SEQ ID NO.: 15) | 20,767,003 | | |
| Aex3F-2 | HLA-A | seq primer | CCCGGTTTCATTTTCAGTTTAGG | * 20,767,061- | 1 μl | 3 μM |
| | | | (SEQ ID NO.: 16) | 20,767,083 | | |
| Aex3R-3 | HLA-A | seq primer | ATTCTAGTGTTGGTCCCAATTGTCTC | * 20,767,502- | 1 μl | 3 μM |
| | | | (SEQ ID NO.: 17) | 20,767,527 | | |
| Aex4F | HLA-A | seq primer | GGTGTCCTGTCCATTCTC | * 20,767,916- | 1 μl | 3 μM |
| | | | (SEQ ID NO.: 18) | 20,767,933 | | |
| Aex4F8001 | HLA-A | seq primer | GGT GTC CTG TCC ATY CTC | * 20,767,916- | 1 μl | 3 μM |
| | | | (SEQ ID NO.: 212) | 20,767,933 | | |
| Aex4R-5 | HLA-A | seq primer | GAGAGGCTCCTGCTTTCCCTA | * 20,768,318- | 1 μl | 3 μM |
| | | | (SEQ ID NO.: 19) | 20,768,338 | | |
| Aex2F-2 | HLA-A | seq primer | GCCTCTGYGGGGAGAAGCAA | * 20,766,542- | 1 μl | 3 μM |
| | | | (SEQ ID NO.: 20) | 20,766,561 | | |
| Aex4R-4 | HLA-A | seq primer | CAGAGAGGCTCCTGCTTTC | * 20,768,322- | 1 μl | 3 μM |
| | | | (SEQ ID NO.: 21) | 20,768,340 | | |

A Locus Multiplex Product Primers

| | | | | | | |
|---|---|---|---|---|---|---|
| pa5-3 | HLA-A | amp primer | CAGACSCCGAGGATGGCC | * 20,766,431- | 0.5 μl | 20 μM |
| | | | (SEQ ID NO.: 1) | 20,766,648 | | |
| pA3-29 | HLA-A | amp primer | GCAGCGACCACAGCTCCAG | * 20,768,461- | 0.5 μl | 20 μM |
| | | | (SEQ ID NO.: 2) | 20,768,479 | | |
| pA5-5 | HLA-A | 5′ amp primer | ACCAGAAGTCGCTGTTCCCTYYTCAGGGA | * 20,767,819- | 0.5 μl | 20 μM |
| | | | (SEQ ID NO.: 3) | 20,767,847 | | |
| pA3-31 | HLA-A | 3′ amp primer | AAAGTCACGGKCCCAAGGCTGCTGCCKGTG | * 20,767,697- | 0.5 μl | 20 μM |
| | | | (SEQ ID NO.: 4) | 20,767,726 | | |
| pA3x23b | HLA-A | 3′ amp primer | CTC AGG ACC AGA GGG AGG GYG | * 20,767,528- | 1.0 μl | 10 μM |
| | | | (SEQ ID No.: 204) | 20,767,550 | | |
| pA3x23b80 | HLA-A | 3′ amp primer | CTC AGG AGC AGA GGG AGG GTG | * 20,767,528- | 1.0 μl | 10 μM |
| | | | (SEQ ID NO.: 205) | 20,767,550 | | |
| pa3-29-2 | HLA-A | amp primer | TCACRGCAGCGACGACAGCTCCAG | * 20,768,456- | 0.5 μl | 20 μM |
| | | | (SEQ ID NO.: 5) | 20,768,479 | | |
| A 3′ UT | HLA-A | amp primer | GCCTTTGCAGAAACAAAGTCAGGGTTC | * 20,769,409- | 0.5 μl | 20 μM |
| | | | (SEQ ID NO.: 6) | 20,769,435 | | |
| pA5-3 + 3 | HLA-A | 5′ amp primer | CCCCAGACSCCGAGGATGGCC | * 20,766,428- | 0.5 μl | 20 μM |
| | | | (SEQ ID NO.: 7) | 20,766,448 | | |
| pA3-31 + 3 | HLA-A | 3′ amp primer | GGAAAAGTCACGGKCCCAAGGCTGCTGCCKGTG | * 20,767,695- | 0.5 μl | 20 μM |
| | | | (SEQ ID NO.: 8) | 20,767,726 | | |
| pA5-9a + 3 | HLA-A | 5′ amp primer | CTTGTTCTGTGCTTCCCACTCAATGTGTG | * 20,767,738- | 0.5 μl | 20 μM |
| | | | (SEQ ID NO.: 9) | 20,767,766 | | |
| pA5-x4a1 | HLA-A | 5′ amp primer | GAC ACA ATT AAG GGA TAA AAT CTC | * 20,767,620- | 1.0 μl | 10 μM |
| | | | TGA AGG AGT GA | 20,767,654 | | |
| | | | (SEQ ID No.: 206) | | | |
| pA5-x4a2 | HLA-A | 5′ amp primer | GAC ACA ATT AAG GGA TAA AAT CTC | * 20,767,620- | 1.0 μl | 10 μM |
| | | | TGA CGG AAT GA | 20,767,654 | | |
| | | | (SEQ ID No.: 207) | | | |
| pA5-x4a3 | HLA-A | 5′ amp primer | GAC ACA ATT AAG GGA TAA AAT CTC | * 20,767,620- | 1.0 μl | 10 μM |
| | | | TGA AGG AAT GA | 20,767,654 | | |
| | | | (SEQ ID No.: 208) | | | |
| pA3-39 + 3 | HLA-A | Ex4 amp primer | GCTGAGATCAGGTCCCATCACTGCCGTA | * 20,768,704- | 0.5 μl | 20 μM |
| | | | (SEQ ID NO.: 10) | 20,768,731 | | |
| pA3-40 + 4 | HLA-A | Ex4 amp primer | GCTGAGATCAGGTCCCATGACCGCTGTA | * 20,768,704- | 0.5 μl | 20 μM |
| | | | (SEQ ID NO.: 11) | 20,768,731 | | |
| pA3-42 + 3 | HLA-A | Ex4 amp primer | GCTGAGATCAGGTCCCATGACCGCCATA | * 20,768,704- | 0.5 μl | 20 μM |
| | | | (SEQ ID NO.: 12) | 20,768,731 | | |
| pA3-43 + 3 | HLA-A | Ex4 amp primer | GCTGAGATCAGGTCCCATCACCGCCGTA | * 20,768,704- | 0.5 μl | 20 μM |
| | | | (SEQ ID NO.: 13) | 20,768,731 | | |
| pA3-43 + 6 | HLA-A | amp primer | ACTGCTAGGATCAGGTCCCATCACCGCCGTA | * 20,768,704- | 1.0 μl | 10 μM |
| | | | (SEQ ID NO.: 22) | 20,768,734 | | |
| pA3-x4b1 | HLA-A | Ex4 amp primer | GGT GCT TCC CAG TAA TGA GAC AGG | * 20,768,171- | 1.0 μl | 10 μM |
| | | | GCA CA | 20,768,199 | | |
| | | | (SEQ ID No.: 209) | | | |
| pA3-x4b2 | HLA-A | Ex4 amp primer | GGT GCT TCC CAG TAA CGA GGC AGG | * 20,768,171- | 1.0 μl | 10 μM |
| | | | GCA CA | 20,768,199 | | |
| | | | (SEQ ID No.: 210) | | | |
| pA3-x4b3 | HLA-A | Ex4 amp primer | GGT GCT TCC CAG GAA TGA GAC AGG | * 20,768,171- | 1.0 μl | 10 μM |
| | | | GCA CA | 20,768,199 | | |
| | | | (SEQ ID No.: 211) | | | |
| pA3-43 + 6a | HLA-A | amp primer | ACTGCTAGGATCAGGTCCCATCACCGCCATA | * 20,768,704- | 1.0 μl | 10 μM |
| | | | (SEQ ID NO.: 23) | 20,768,734 | | |
| pA3-43 + 6b | HLA-A | amp primer | ACTGCTAGGATCAGGTCCCATCACCGCTGTA | * 20,768,704- | 1.0 μl | 10 μM |
| | | | (SEQ ID NO.: 24) | 20,768,734 | | |
| pA3-43 + 6c | HLA-A | amp primer | ACTGCTAGGATCAGGTCCCATCACTGCCGTA | * 20,768,704- | 1.0 μl | 10 μM |
| | | | (SEQ ID NO.: 25) | 20,768,734 | | |
| pA5-9 + 8 | HLA-A | amp primer | CAGGCCTTGTTCTCTGCTTCACACTCAATGTGTG | * 20,767,733- | 0.5 μl | 2O μM |
| | | | (SEQ ID NO.: 26) | 20,767,766 | | |
| pA3-52 | HLA-A | amp primer | CAGGGCCTTAAGGTCCTAGAGGAACCTCC | * 20,768,880- | 0.5 μl | 20 μM |
| | | | (SEQ ID NO.: 27) | 20,768,907 | | |
| pA3-50-1 | HLA-A | amp primer | GAACCTGGTCAGATCCCACAGAASATGTGGC | * 20,769,073- | 0.5 μl | 20 μM |
| | | | (SEQ ID NO.: 28) | 20,769,103 | | |
| pA3-53a | HLA-A | amp primer | TGGGTGAGCTCCCCCATGGGCTCC | * 20,769,030- | 0.5 μl | 20 μM |
| | | | (SEQ ID NO.: 29) | 20,769,049 | | |
| pA3-53b | HLA-A | amp primer | TGGGTGGGCTCCCCCATGGGCTCC | * 20,769,030- | 0.5 μl | 20 μM |
| | | | (SEQ ID NO.: 30) | 20,769,049 | | |
| pA3-53c | HLA-A | amp primer | TGGTTGAGCTCCCCCATGGGCTCC | * 20,769,030- | 0.5 μl | 20 μM |
| | | | (SEQ ID NO.: 31) | 20,769,049 | | |
| pA3-53d | HLA-A | amp primer | TGGGTGAGCTCCCCCACGGGCTCC | * 20,769,030- | 0.5 μl | 20 μM |
| | | | (SEQ ID NO.: 32) | 20,769,049 | | |
| pA3-31b + 3 | HLA-A | amp primer | GGAAAAGTCACGGGCCCAAGGCTGCTGCCKGTG | * 20,767,695- | 0.5 μl | 20 μM |
| | | | (SEQ ID NO.: 33) | 20,767,726 | | |
| A3′UT-2 | HLA-A | amp primer | CAGGTGCCTTTGCAGAAACAAAGTCAGGGT | * 20,769,409- | 0.5 μl | 20 μM |
| | | | (SEQ ID NO.: 34) | 20,769,440 | | |
| pA5-8 + 6 | HLA-A | amp primer | CACGGAATAGRAGATTATCCCAGGTGCCT | * 20,767,842- | 0.5 μl | 20 μM |
| | | | (SEQ ID NO.: 35) | 20,767,870 | | |
| Aex2F | HLA-A | seq primer | GGGAAACSGCCTCTG | * 20,766,534- | 0.5 μl | 20 μM |
| | | | (SEQ ID NO.: 14) | 20,766,548 | | |
| Aex2R-4 | HLA-A | seq primer | GGATCTCGGACCCGGAGACTGT | * 20,766,982- | 1 μl | 3 μM |
| | | | (SEQ ID NO.: 15) | 20,767,003 | | |
| Aex3F-2 | HLA-A | seq primer | CCCGGTTTCATTTTCAGTTTAGG | * 20,767,061- | 1 μl | 3 μM |
| | | | (SEQ ID NO.: 16) | 20,767,083 | | |
| Aex3R-3 | HLA-A | seq primer | ATTCTAGTGTTGGTCCCAATTGTCTC | * 20,767,502- | 1 μl | 3 μM |
| | | | (SEQ ID NO.: 17) | 20,767,527 | | |
| Aex4F | HLA-A | seq primer | GGTGTCCTGTCCATTCTC | * 20,767,916- | 1 μl | 3 μM |
| | | | (SEQ ID NO.: 18) | 20,767,933 | | |
| Aex4R-5 | HLA-A | seq primer | GAGAGGCTCCTGCTTTCCCTA | * 20,768,318- | 1 μl | 3 μM |
| | | | (SEQ ID NO.: 19) | 20,768,338 | | |
| Aex2F-2 | HLA-A | seq primer | GCCTCTGYGGGGAGAAGCAA | * 20,766,542- | 1 μl | 3 μM |
| | | | (SEQ ID NO.: 20) | 20,766,561 | | |
| Aex4R-4 | HLA-A | seq primer | CAGAGAGGCTCCTGCTTTC | * 20,768,322- | 1 μl | 3 μM |
| | | | (SEQ ID NO.: 21) | 20,768,348 | | |
| Aex4F8001 | HLA-A | seq primer | GGT GTC CTG TCC ATY CTC | * 20,767,916- | 1 μl | 3 μM |
| | | | (SEQ ID NO.: 212) | 20,767,933 | | |

B Locus Multiplex Product Primers

| | | | | | | |
|---|---|---|---|---|---|---|
| pB3-24 | HLA-B | 3′ amp primer | GGTKCCCAAGGCTGCTGCAGGGG | * 22,178,140- | 0.5 μl | 20 μM |
| | | | (SEQ ID NO.: 36) | 22,178,162 | | |
| pB5-48 | HLA-B | amp primer | GAACCGTCCTCCTGCTGCTCTC | * 22,179,358- | 0.5 μl | 20 μM |
| | | | (SEQ ID NO.: 37) | 22,179,379 | | |
| pB5-49 | HLA-B | amp primer | GAACCGTCCTCCTGCTGCTCTG | * 22,179,358- | 0.5 μl | 20 μM |
| | | | (SEQ ID NO.: 38) | 22,179,379 | | |
| pB3-20 | HLA-B | 3′ amp primer | ATCACAGCAGCGACCACAGCTCCGAT | * 22,177,368- | 0.5 μl | 10 μM |
| | rev | | (SEQ ID NO.: 39) | 22,177,393 | | |
| pB3-21 | HLA-B | 3′ amp primer | ATCACAGTAGCGACCACAGCTCCGAT | * 22,177,368- | 0.5 μl | 10 μM |
| | rev | | (SEQ ID NO.: 40) | 22,177,393 | | |
| pB3-22 | HLA-B | 3′ amp primer | ATCACAGTAGCAACCACAGCTCCGAT | * 22,177,368- | 0.5 μl | 10 μM |
| | rev | | (SEQ ID NO.: 41) | 22,177,393 | | |
| pB3-23 | HLA-B | 3′ amp primer | ATCACAGCAGCGACCACAGCGACCAC | * 22,177,368- | 0.5 μl | 10 μM |
| | rev | | (SEQ ID NO.: 42) | 22,177,393 | | |
| pB5-55 + 4 | HLA-B | 5′ amp primer | GGCTCTGATTCCAGCACTTCTGAGTCACTTTC | * 22,178.056- | 0.5 μl | 20 μM |
| | | | (SEQ ID NO.: 43) | 22,178,078 | | |
| pB5-52 | HLA-B | 5′ amp primer | GACCACAGGCTGGGGCGCAGGACCCGG | * 22,179,251- | 0.5 μl | 20 μM |
| | | | (SEQ ID NO.: 44) | 22,179,277 | | |
| pB5-53 | HLA-B | 5′ amp primer | GACCACAGGCGGGGGCGCAGGACCTGA | * 22,179,251- | 0.5 μl | 20 μM |
| | | | (SEQ ID NO.: 45) | 22,179,277 | | |
| pB5-44 | HLA-B | 5′ amp primer | ACGCACCCACCCGGACTCAGAA | * 22,179,416- | 0.5 μl | 20 μM |
| | | | (SEQ ID NO.: 46) | 22,179,437 | | |
| pB5-45 | HLA-B | 5′ amp primer | ACGCACCCACCCGGACTCAGAG | * 22,179,416- | 0.5 μl | 20 μM |
| | | | (SEQ ID NO.: 47) | 22,179,437 | | |
| B 3′UT | HLA-B | 3′ amp primer | AGAGGCTCTTGAAGTCACAAAGGGGA | * 22,176,462- | 0.5 μl | 20 μM |
| | | | (SEQ ID NO.: 48) | 22,176,487 | | |
| pB5-48a | HLA-B | 5′ amp primer | ACTGTGAACCGTCCTCCTGCTGCTCTC | * 22,179,353- | 0.5 μl | 20 μM |
| | | | (SEQ ID NO.: 49) | 22,179,379 | | |
| pB5-49 + 1Ca | HLA-B | 5′ amp primer | AAGTGCGAACCCTCCTCCTGCTGCTCTG | * 22,179,352- | 0.5 μl | 20 μM |
| | | | (SEQ ID NO.: 50) | 22,179,379 | | |
| pB5-49 + 1a | HLA-B | 5′ amp primer | AAGTGCGAACCGTCCTCCTGCTGCTCTG | * 22,179,352- | 0.5 μl | 20 μM |
| | | | (SEQ ID NO.: 51) | 22,179,379 | | |
| pB3-24a | HLA-B | 3′ amp primer | ACTGCGGTKCCCAAGGCTGCTGCAGGGG | * 22,178,135- | 0.5 μl | 20 μM |
| | | | (SEQ ID NO.: 52) | 22,178,162 | | |
| yB2F-6a + 10 | HLA-B | seq primer | ATTATGATTAAGCCCCTCCTCRCCCCCAG | * 22,179,198- | 1 μl | 3 μM |
| | | | (SEQ ID NO.: 53) | 22,179,216 | | |
| yB2F-5a + 10 | HLA-B | seq primer | ATTATGATTACAGCCCCTCCTTGCCCCAG | * 22,179,197- | 1 μl | 3 μM |
| | | | (SEQ ID NO.: 54) | 22,179,216 | | |
| yB2F-12a + 10 | HLA-B | seq primer | ATTATGATTAAGCCCCTCCTGGCCCCCAG | * 22,179,198- | 1 μl | 3 μM |
| | | | (SEQ ID NO.: 55) | 22,179,216 | | |
| yB2R-4 | HLA-B | seq primer | GGAGGGGTCGTGACCTGCG | * 22,178,886- | 1 μl | 3 μM |
| | | | (SEQ ID NO.: 56) | 22,178,906 | | |
| yB3F-2a + 10 | HLA-B | seq primer | ATTATGATTAGGGGACGGGGCTGACC | * 22,178,698- | 1 μl | 3 μM |
| | | | (SEQ ID NO.: 57) | 22,178,712 | | |
| yB3F-2b + 10 | HLA-B | seq primer | ATTATGATTAGGGGACTGGGCTGACC | * 22,178,698- | 1 μl | 3 μM |
| | | | (SEQ ID NO.: 58) | 22,178,712 | | |
| yB3F-2c + 10 | HLA-B | seq primer | ATTATGATTAGGGGACGGTGCTGACC | * 22,178,698- | 1 μl | 3 μM |
| | | | (SEQ ID NO.: 59) | 22,178,712 | | |
| B-Ex3R | HLA-B | seq primer | AAACTCATGCCATTCTCCATTC | * 22,178,276- | 1 μl | 3 μM |
| | | | (SEQ ID NO.: 60) | 22,178,297 | | |
| B-Ex4F1 | HLA-B | seq primer | GTCACATGGGTGGTCCTA | * 22,177,887- | 1 μl | 3 μM |
| | | | (SEQ ID NO.: 61) | 22,177,904 | | |
| yB4R-3 | HLA-B | seq primer | GGCTCCTGCTTTCCCTGAGAA | * 22,177,508- | 1 μl | 3 μM |
| | | | (SEQ ID NO.: 62) | 22,177,738 | | |
| yB2F-6b + 10 | HLA-B | seq primer | ATTATGATTACCCCTCCTCRCCCCCAG | * 22,179,200- | 1 μl | 3 μM |
| | | | (SEQ ID NO.: 63) | 22,179,216 | | |
| yB2F-5b + 10 | HLA-B | seq primer | ATTATGATTAGCCCCTCCTTGCCCCAG | * 22,179,199- | 1 μl | 3 μM |
| | | | (SEQ ID NO.: 64) | 22,179,216 | | |
| yB2F-12b + 10 | HLA-B | seq primer | ATTATGATTACCCCTCCTGGCCCCCAG | * 22,179,200- | 1 μl | 3 μM |
| | | | (SEQ ID NO.: 65) | 22,179,216 | | |
| yB2F-19b + 10 | HLA-B | seq primer | ATTATGATTACCCCTCCTCGCTCCCAG | * 22,179,200- | 1 μl | 3 μM |
| | | | (SEQ ID NO.: 66) | 22,179,216 | | |
| yB2F-6c + 10 | HLA-B | seq primer | ATTATGATTACCTCCTCRCCCCCAG | * 22,179,202- | 1 μl | 3 μM |
| | | | (SEQ ID NO.: 67) | 22,179,216 | | |
| yB2F-5c + 10 | HLA-B | seq primer | ATTATGATTACCCTCCTTGCCCCAG | * 22,179,201- | 1 μl | 3 μM |
| | | | (SEQ ID NO.: 68) | 22,179,216 | | |
| yB2F-12c + 10 | HLA-B | seq primer | ATTATGATTACCTCCTGGCCCCCAG | * 22,179,202- | 1 μl | 3 μM |
| | | | (SEQ ID NO.: 69) | 22,179,216 | | |
| yB2F-19c + 10 | HLA-B | seq primer | ATTATGATTACCTCCTCGCTCCCAG | * 22,179,202- | 1 μl | 3 μM |
| | | | (SEQ ID NO.: 70) | 22,179,216 | | |
| yB2F-5a | HLA-B | seq primer | CAGCCCCTCCTTGCCCCAG | * 22,179,196- | 1 μl | 3 μM |
| | | | (SEQ ID NO.: 71) | 22,179,216 | | |
| yB2F-6a | HLA-B | seq primer | AGCCCCTCCTCRCCCCCAG | * 22,179,196- | 1 μl | 3 μM |
| | | | (SEQ ID NO.: 72) | 22,179,216 | | |
| yB2F-7a | HLA-B | seq primer | AGCTCCTCCTCGCCCCCAG | * 22,179,196- | 1 μl | 3 μM |
| | | | (SEQ ID NO.: 73) | 22,179,216 | | |
| yB2F-12a | HLA-B | seq primer | AGCCCCTCCTGGCCCCCAG | * 22,179,196- | 1 μl | 3 μM |
| | | | (SEQ ID NO.: 74) | 22,179,216 | | |
| yB3F-2a | HLA-B | seq primer | GGGGACGGGGCTGACC | * 22,178,698- | 1 μl | 3 μM |
| | | | (SEQ ID NO.: 75) | 22,178,712 | | |
| yB3F-2b | HLA-B | seq primer | GGGGACTGGGCTGACC | * 22,178,698- | 1 μl | 3 μM |
| | | | (SEQ ID NO.: 76) | 22,178,712 | | |
| yB3F-2c | HLA-B | seq primer | GGGGACGGTGCTGACC | * 22,178,698- | 1 μl | 3 μM |
| | | | (SEQ ID NO.: 77) | 22,178,712 | | |

B Locus Single Product Primers

| | | | | | | |
|---|---|---|---|---|---|---|
| pB5-48 | HLA-B | 5′ amp primer | GAACCGTCCTCCTGCTGCTCTC | * 22,179,358- | 0.5 μl | 20 μM |
| | | | (SEQ ID NO.: 37) | 22,179,379 | | |
| pB5-49 | HLA-B | 5′ amp primer | GAACCGTCCTCCTGCTGCTCTG | * 22,179,358- | 0.5 μl | 20 μM |
| | | | (SEQ ID NO.: 38) | 22,179,379 | | |
| pB3-20 | HLA-B | 3′ amp primer | ATCACAGCAGCGACCACAGCTCCGAT | * 22,177,368- | 0.5 μl | 20 μM |
| | | | (SEQ ID NO.: 39) | 22,177,393 | | |
| pB3-21 | HLA-B | 3′ amp primer | ATCACAGTAGCGACCACAGCTCCGAT | * 22,177,368- | 0.5 μl | 20 μM |
| | | | (SEQ ID NO.: 40) | 22,177,393 | | |
| pB3-22 | HLA-B | 3′ amp primer | ATCACAGTAGCAACCACAGCTCCGAT | * 22,177,368- | 0.5 μl | 20 μM |
| | | | (SEQ ID NO.: 41) | 22,177,393 | | |
| pB3-23 | HLA-B | 3′ amp primer | ATCACAGCAGCGACCACAGCGACCAC | * 22,177,368- | 0.5 μl | 20 μM |
| | | | (SEQ ID NO.: 42) | 22,177,393 | | |
| pB5-55 + 4 | HLA-B | 5′ amp primer | GGCTCTGATTCCAGCACTTCTGAGTCACTTTAC | * 22,178,056- | 0.5 μl | 20 μM |
| | | | (SEQ ID NO.: 43) | 22,178,078 | | |
| pB3-24 | HLA-B | 3′ amp primer | GGTKCCCAAGGCTGCTGCAGGGG | * 22,178,140- | 0.5 μl | 20 μM |
| | | | (SEQ ID NO.: 36) | 22,178,162 | | |
| yB2F-6a + 10 | HLA-B | seq primer | ATTATGATTAAGCCCCTCCTCRCCCCCAG | * 22,179,198- | 1 μl | 3 μM |
| | | | (SEQ ID NO.: 53) | 22,179,216 | | |
| yB2F-5a + 10 | HLA-B | seq primer | ATTATGATTACAGCCCCTCCTTGCCCCAG | * 22,179.197- | 1 μl | 3 μM |
| | | | (SEQ ID NO.: 54) | 22,179,216 | | |
| yB2F-12a + 10 | HLA-B | seq primer | ATTATGATTAAGCCCCTCCTGGCCCCCAG | *22,179,198 | 1 μl | 3 μM |
| | | | (SEQ ID NO.: 55) | 22,179,216 | | |
| yB2R-4 | HLA-B | seq primer | GGAGGGGTCGTGACCTGCG | *22,178,886- | 1 μl | 3 μM |
| | | | (SEQ ID NO.: 56) | 22,178,906 | | |
| yB3F-2a + 10 | HLA-B | seq primer | ATTATGATTAGGGGACGGGGCTGACC | *22,178,698- | 1 μl | 3 μM |
| | | | (SEQ ID NO.: 57) | 22,178,712 | | |
| yB3F-2b + 10 | HLA-B | seq primer | ATTATGATTAGGGGACTGGGCTGACC | *22,178,698 | 1 μl | 3 μM |
| | | | (SEQ ID NO.: 58) | 22,178,712 | | |
| yB3F-2c + 10 | HLA-B | seq primer | ATTATGATTAGGGGACGGTGCTGACC | * 22,178,698- | 1 μl | 3 μM |
| | | | (SEQ ID NO.: 59) | 22,178,712 | | |
| B-Ex3R | HLA-B | seq primer | AAACTCATGCCATTCTCCATTC | * 22,178,276- | 1 μl | 3 μM |
| | | | (SEQ ID NO.: 60) | 22,178,297 | | |
| B-Ex4F1 | HLA-B | seq primer | GTCACATGGGTGGTCCTA | * 22,177,887- | 1 μl | 3 μM |
| | | | (SEQ ID NO.: 61) | 22,177,904 | | |
| yB4R-3 | HLA-B | seq primer | GGCTCCTGCTTTCCCTGAGAA | * 22,177,508- | 1 μl | 3 μM |
| | | | (SEQ ID NO.: 62) | 22,177,738 | | |
| yB2F-5a | HLA-B | seq primer | CAGCCCCTCCTTGCCCCAG | * 22,179,196- | 1 μl | 3 μM |
| | | | (SEQ ID NO.: 71) | 22,179,216 | | |
| yB2F-6a | HLA-B | seq primer | AGCCCCTCCTCRCCCCCAG | * 22,179,196- | 1 μl | 3 μM |
| | | | (SEQ ID NO.: 72) | 22,179,216 | | |
| yB2F-7a | HLA-B | seq primer | AGCTCCTCCTCGCCCCCAG | * 22,179,196- | 1 μl | 3 μM |
| | | | (SEQ ID NO.: 73) | 22,179,216 | | |
| yB2F-12a | HLA-B | seq primer | AGCCCCTCCTGGCCCCCAG | * 22,179,196- | 1 μl | 3 μM |
| | | | (SEQ ID NO.: 74) | 22,179,216 | | |
| yB3F-2a | HLA-B | seq primer | GGGGACGGGGCTGACC | * 22,178,698- | 1 μl | 3 μM |
| | | | (SEQ ID NO.: 75) | 22,178,712 | | |
| yB3F-2b | HLA-B | seq primer | GGGGACTGGGCTGACC | * 22,178,698- | 1 μl | 3 μM |
| | | | (SEQ ID NO.: 76) | 22,178,712 | | |
| yB3F-2c | HLA-B | seq primer | GGGGACGGTGCTGACC | * 22,178,698- | 1 μl | 3 μM |
| | | | (SEQ ID NO.: 77) | 22,178,712 | | |
| yB2F-6b + 10 | HLA-B | seq primer | ATTATGATTACCCCTCCTCRCCCCCAG | * 22,179,200- | 1 μl | 3 μM |
| | | | (SEQ ID NO.: 63) | 22,179,216 | | |
| yB2F-5b + 10 | HLA-B | seq primer | ATTATGATTAGCCCCTCCTTGCCCCAG | * 22,179,199- | 1 μl | 3 μM |
| | | | (SEQ ID NO.: 64) | 22,179,216 | | |
| yB2F-12b + 10 | HLA-B | seq primer | ATTATGATTACCCCTCCTGGCCCCCAG | * 22,179,200- | 1 μl | 3 μM |
| | | | (SEQ ID NO.: 65) | 22,179,216 | | |
| yB2F-19b + 10 | HLA-B | seq primer | ATTATGATTACCCCTCCTCGCTCCCAG | * 22,179,200- | 1 μl | 3 μM |
| | | | (SEQ ID NO.: 66) | 22,179,216 | | |
| yB2F-6c + 10 | HLA-B | seq primer | ATTATGATTACCTCCTCRCCCCCAG | * 22,179,202- | 1 μl | 3 μM |
| | | | (SEQ ID NO.: 67) | 22,179,216 | | |
| yB2F-5c + 10 | HLA-B | seq primer | ATTATGATTACCCTCCTTGCCCCAG | * 22,179,201- | 1 μl | 3 μM |
| | | | (SEQ ID NO.: 68) | 22,179,216 | | |
| yB2F-12c + 10 | HLA-B | seq primer | ATTATGATTACCTCCTGGCCCCCAG | * 22,179,202- | 1 μl | 3 μM |
| | | | (SEQ ID NO.: 69) | 22,179,216 | | |
| yB2F-19c + 10 | HLA-B | seq primer | ATTATGATTACCTCCTCGCTCCCAG | * 22,179,202- | 1 μl | 3 μM |
| | | | (SEQ ID NO.: 70) | 22,179,216 | | |

C Locus Single Product Primers

| | | | | | | |
|---|---|---|---|---|---|---|
| C Intron 3 R | HLA-C | amp primer | GCAGTGGTCAAAGTGGTCA | * 22,093,610- | 0.75 μl | 20 μM |
| | | | (SEQ ID NO.: 78) | 22,093,628 | | |
| C Intron 3 F | HLA-C | amp primer | GCAGCTGTGGTCAGGCTGCT | * 22,093,589- | 0.75 μl | 20 μM |
| | | | (SEQ ID NO.: 79) | 22,093,608 | | |
| C 3′ UT | HLA-C | amp primer | GGACACGGGGGTGRGCTGTCTSTC | * 22,091,807- | 0.75 μl | 20 μM |
| | | | (SEQ ID NO.: 80) | 22,091,830 | | |
| C5ApUTG | HLA-C | amp primer | CAGTCCCGGTTCTGAAGTCCCCAGT | * 22,094,905- | 0.75 μl | 20 μM |
| | | | (SEQ ID NO.: 81) | 22,094,929 | | |
| C5ApUTA | HLA-C | amp primer | CAGTCCCGGTTCTAAAGTCCCCAGT | * 22,094,905- | 0.75 μl | 20 μM |
| | | | (SEQ ID NO.: 82) | 22,094,929 | | |
| C5X1_I1GG | HLA-C | amp primer | GGGCCGGTGAGTGCGGGGTT | * 22,094,782- | 1.5 μl | 10 μM |
| | | | (SEQ ID NO.: 83) | 22,094,801 | | |
| C5X1_I1TA | HLA-C | amp primer | GGGCCTGTGAGTGCGAGGTT | * 22,094,782- | 1.5 μl | 10 μM |
| | | | (SEQ ID NO.: 84) | 22,094,801 | | |
| C5X1_I1TG | HLA-C | amp primer | GGGCCTGTGAGTGCGGGGTT | * 22,094,782- | 1.5 μl | 10 μM |
| | | | (SEQ ID NO.: 85) | 22,094,801 | | |
| C3ApX5A | HLA-C | amp primer | AGCTCCAAGGACAGCTAGGACA | * 22,092,800- | 1.5 μl | 10 μM |
| | | | (SEQ ID NO.: 86) | 22,092,821 | | |
| C3ApX5T | HLA-C | amp primer | AGCTCCTAGGACAGCTAGGACA | * 22,092,800- | 1.5 μl | 10 μM |
| | | | (SEQ ID NO.: 87) | 22,092,821 | | |
| C173ApX5 | HLA-C | amp primer | GACAGCCAGGACAGCCAGGACA | * 22,092,800- | 0.75 μl | 20 μM |
| | | | (SEQ ID NO.: 88) | 22,092,821 | | |
| C3ApI4T | HLA-C | amp primer | GTGAGGGGCCCTGACCTCCAA | * 22,092,901- | 1.5 μl | 10 μM |
| | | | (SEQ ID NO.: 89) | 22,092,921 | | |
| C3ApI4C | HLA-C | amp primer | GTGAGGGGCCCTGACCCCCAA | * 22,092,901- | 1.5 μl | 10 μM |
| | | | (SEQ ID NO.: 90) | 22,092,921 | | |
| C3ApI4TAC | HLA-C | amp primer | GTGAGGGGCCCTTACACCCAA | * 22,092,901- | 1.5 μl | 10 μM |
| | | | (SEQ ID NO.: 91) | 22,092,921 | | |
| CApExon5R2 | HLA-C | amp primer | GCCATCACAGCTCCTAGGACAGCTA | * 22,092,792- | 1.5 μl | 10 μM |
| | | | (SEQ ID NO.: 92) | 22,092,816 | | |
| CApExon5R3 | HLA-C | amp primer | GCCACCATAGCTCCTAGGACAGCTA | * 22,092,792- | 1.5 μl | 10 μM |
| | | | (SEQ ID NO.: 93) | 22,092,816 | | |
| CApExon5R4 | HLA-C | amp primer | GTGACCACAGCTCCAAGGACAGCTA | * 22,092,792- | 1.5 μl | 10 μM |
| | | | (SEQ ID NO.: 94) | 22,092,816 | | |
| CApExon5R5 | HLA-C | amp primer | AGCTAGGACAGCCAGGACAGCCA | * 22,092,792- | 1.5 μl | 10 μM |
| | | | (SEQ ID NO.: 95) | 22,092,816 | | |
| CApExon5R1 | HLA-C | amp primer | CCACCACAGCTCCTAGGACAGCTA | * 22,092,792- | 1.5 μl | 10p μ |
| | | | (SEQ ID NO.: 96) | 22,092,816 | | |
| pC5-2 | HLA-C | amp primer | CAGTCCCGGTTCTRAAGTCCCCAGT | * 22,094,905- | 0.75 μl | 20 μM |
| | | | (SEQ ID NO.: 97) | 22,094,929 | | |
| C5′UT | HLA-C | amp primer | CCACTCCCATTGGGTGTCGGRTTCT | * 22,094,953- | 0.75 μl | 20 μM |
| | | | (SEQ ID NO.: 98) | 22,094,977 | | |
| C-13R | HLA-C | amp primer | CCACAGCTGCYGCAGTAGTCAAAGTGGTC | * 22,093,599- | 0.75 μl | 20 μM |
| | | | (SEQ ID NO.: 99) | 22,093,627 | | |
| C-13F-2 | HLA-C | amp primer | CTCAGGTCAGGACCAGAAGTCGCTGTTCAT | * 22,093,473- | 0.75 μl | 20 μM |
| | | | (SEQ ID NO.: 100) | 22,093,502 | | |
| PC3-I52196G | HLA-C | amp primer | CTGAGATGGCCCAGGTGTGGATGG | * 22,092,643- | 1.5 μl | 10 μM |
| | | | (SEQ ID NO.: 101) | 22,092,666 | | |
| PC3-I52196T | HLA-C | amp primer | CTGAGATGGCCCATGTGTGGATGG | * 22,092,643- | 1.5 μl | 10 μM |
| | | | (SEQ ID NO.: 102) | 22,092,666 | | |
| c5x21 | HLA-C | seq primer | GGAGCCGCGCAGGGAGG | * 22,094,702- | 1 μl | 3 μM |
| | | | (SEQ ID NO.: 103) | 22,094,718 | | |
| 5x22 | HLA-C | seq primer | GGGTCGGGCGGGTCTCAG | * 22,094,681- | 1 μl | 3 μM |
| | | | (SEQ ID NO.: 104) | 22,094,700 | | |
| c3x21 | HLA-C | seq primer | GGCCGTCCGTGGGGGATG | * 22,094,336- | 1 μl | 3 μM |
| | | | (SEQ ID NO.: 105) | 22,094,354 | | |
| c3x22 | HLA-C | seq primer | TCGKGACCTGCGCCCGG | * 22,094,363- | 1 μl | 3 μM |
| | | | (SEQ ID NO.: 106) | 22,094,379 | | |
| c5x31 | HLA-C | seq primer | TTCRGTTTAGGCCAAAATCCCCGC | * 22,094,205- | 1 μl | 3 μM |
| | | | (SEQ ID NO.: 107) | 22,094,228 | | |
| c5x32 | HLA-C | seq primer | GTCRCCTTTACCCGGTTTCATTTTC | * 22,094,226- | 1 μl | 3 μM |
| | | | (SEQ ID NO.: 108) | 22,094,250 | | |
| c3x31 | HLA-C | seq primer | GCTGATCCCATTTTCCTCCCCTCC | * 22,093,783- | 1 μl | 3 μM |
| | | | (SEQ ID NO.: 109) | 22,093,806 | | |
| c5x41 | HLA-C | seq primer | AGGCTGGCGTCTGGGTTCTGTG | * 22,093,395- | 1 μl | 3 μM |
| | | | (SEQ ID NO.: 110) | 22,093,415 | | |
| c5x42 | HLA-C | seq primer | CCRTTCTCAGGATRGTCACATGGGC | * 22,093,343- | 1 μl | 3 μM |
| | | | (SEQ ID NO.: 111) | 22,093,367 | | |
| c5x43 | HLA-C | seq primer | CAAAGTGTCTGAATTTTCTGACTCTTCCC | * 22,093,288- | 1 μl | 3 μM |
| | | | (SEQ ID NO.: 112) | 22,093,316 | | |
| c3x41 | HLA-C | seq primer | AGGACTTCTGCTTTCYCTGAKAAG | * 22,092,955- | 1 μl | 3 μM |
| | | | (SEQ ID NO.: 113) | 22,092,978 | | |
| c5x21 + 15 | HLA-C | seq primer | ATGATATTATGATTAGGAGCCGCGCAGGGAGG | * 22,094,702- | 1 μl | 3 μM |
| | | | (SEQ ID NO.: 114) | 22,094,720 | | |
| c5x3_14 + 10 | HLA-C | seq primer | ATTATGATTACTCGGGGGACGGGGCTGACC | * 22,094,162- | 1 μl | 3 μM |
| | | | (SEQ ID NO.: 115) | 22,094,181 | | |
| c3x41_3 + 7 | HLA-C | seq primer | ATGATTAACCCCTCATCCCCCTCCTTA | * 22,092,987- | 1 μl | 3 μM |
| | | | (SEQ ID NO.: 116) | 22,093,005 | | |
| c3x41_4 + 7 | HLA-C | seq primer | ATGATTAACCCCCCATTCCCCTCCTTA | * 22,092,987- | 1 μl | 3 μM |
| | | | (SEQ ID NO.: 117) | 22,093,005 | | |
| c3x41_3 + 15 | HLA-C | seq primer | ATGATATTATGATTAACCCCTCATCCCCCTCCT | * 22,092,987- | 1 μl | 3 μM |
| | | | TA | 22,092,005 | | |
| | | | (SEQ ID NO.: 118) | | | |
| c3x41_4 + 15 | HLA-C | seq primer | ATGATATTATGATTAACCCCCCATTCCCCTCCT | * 22,092,987- | 1 μl | 3 μM |
| | | | TA | 22,093,005 | | |
| | | | (SEQ ID NO.: 119) | | | |

DRB Locus Single Tube Multiplex Primers

| | | | | | | |
|---|---|---|---|---|---|---|
| OTDR-01 | DRB1 | 5′ amp primer | TGTAAAACGACGGCCAGTCCCACAGCACGTTTCT | * 23,354,395- | 1.7 μl | 10 μM |
| | | | TGTG | 23,354,415 | | |
| | | | (SEQ ID NO.: 120) | | | |
| OTDR- | DRB1 | 5′ amp primer | TGTAAAACGACGGCCAGTCCCACAGCACGTTTCC | * 23,354,396- | 1.1 μl | 10 μM |
| 02/07 | | | TGT | 23,354,415 | | |
| | | | (SEQ ID NO.: 121) | | | |
| OTDR- | DRB1 | 5′ amp primer | TGTAAAACGACGGCCAGTTTCACAGCACGTTTCT | * 23,354,391- | 3.9 μl | 10 μM |
| 03/5/6/08/12 | | | TGGAGTAC | 23,354,414 | | |
| | | | (SEQ ID NO.: 122) | | | |
| OTDR-04 | DRB1 | 5′ amp primer | TGTAAAACGACGGCCAGTTACTAATCACGTTTCT | * 23,354,389- | 4.6 μl | 10 μM |
| | | | TGGAGCAGGT | 23,354,407 | | |
| | | | (SEQ ID NO.: 123) | | | |
| OTDR-09 | DRB1 | 5′ amp primer | TGTAAAACGACGGCCAGTTCCACAGCACGTTTCT | * 23,354,396- | 28.0 μl | 10 μM |
| | | | TGA | 23,354,414 | | |
| | | | (SEQ ID NO.: 124) | | | |
| OTDR-10 | DRB1 | 5′ amp primer | TGTAAAACGACGGCCAGTTACTAATCACGTTTCT | * 23,354,390- | 2.92 μl | 10 μM |
| | | | TGGAGGAGG | 23,354,409 | | |
| | | | (SEQ ID NO.: 125) | | | |
| OTDR-04-5 | HLA- | 5′ amp primer | TGTAAAACGACGGCCAGTTACTAATCACGTTTCT | * 23,354,384- | 4.6 μl | 10 μM |
| | DRB | | TGGAGCAGGTTAAAC | 23,354,408 | | |
| | | | (SEQ ID NO.: 126) | | | |
| OTDR-10-4 | HLA- | 5′ amp primer | TGTAAAACGACGGCCAGTATCACAGCACGTTTCT | * 23,354,390- | 2.92 μl | 10 μM |
| | DRB | | TGGAGG | 23,354,413 | | |
| | | | (SEQ ID NO.: 127) | | | |
| OTDR-09-2 | HLA- | 5′ amp primer | TGTAAAACGACGGCCAGTTACTAATCACGTTTCT | * 23,354,383- | 28.0 μl | 10 μM |
| | DRB | | TGAAGCAGGATAAGTT | 23,354,408 | | |
| | | | (SEQ ID NO.: 128) | | | |
| OTDR-3-2 | HLA- | 3′ amp primer | CAGGAAACAGCTATGACCCRYGCTYACCTCGCCK | * 23,354,129- | 0.6 μl | 10 μM |
| | DRB | | CTG | 23,354,147 | | |
| | | | (SEQ ID NO.: 129) | | | |
| OTDR-09-8 | HLA- | 5′ amp primer | TGTAAACGACGGCCAGTTACTAATTGTGTTTCTT | * 23,354,383- | 28.0 μl | 10 μM |
| | DRB | | GAAGCAGGATAAGTT | 23,354,408 | | |
| | | | (SEQ ID NO.: 130) | | | |
| M13 | | seq primer | TGTAAAACGACGGCCAGT | N/A | 1 μl | 3 μM |
| Forward | | | (SEQ ID NO.: 131) | | | |
| M13 | | seq primer | CAGGAAACAGCTATGACC | N/A | 1 μl | 3 μM |
| Reverse | | | (SEQ ID NO.: 132) | | | |

DRB Locus Group Specific Multiplex Primers

| | | | | | | |
|---|---|---|---|---|---|---|
| GSDR-01 | HLA-DRB | 5′ amp primer | TGTAAAACGACGGCCAGTCACGTTTCTTGTGGSA | * 23,354,388- | 0.6 μl | 10 μM |
| | | | GCTT | 23,354,407 | | |
| | | | (SEQ ID NO.: 133) | | | |
| GSDR- | HLA-DRB | 5′ amp primer | TGTAAAACGACGGCCAGTTTCCTGTGGCAGCCTA | * 23,354,384- | 0.6 μl | 10 μM |
| 15/16 | | | AGA | 23,354,402 | | |
| | | | (SEQ ID NO.: 134) | | | |
| GSDR- | HLA-DRB | 5′ amp primer | TGTAAAACGACGGCCAGTCGTTTCTTGGAGTACT | * 23,354,383- | 0.6 μl | 10 μM |
| 03/11/13/14 | | | CTACGTC | 23,354,405 | | |
| | | | (SEQ ID NO.: 135) | | | |
| GSDR-04 | HLA-DRB | 5′ amp primer | TGTAAAACGACGGCCAGTCGTTTCTTGGAGCAGG | * 23,354,384- | 0.6 μl | 10 μM |
| | | | TTAAAC | 23,354,405 | | |
| | | | (SEQ ID NO.: 136) | | | |
| GSDR-07 | HLA-DRB | 5′ amp primer | TGTAAAACGACGGCCAGTTTCCTGTGGCAGGGTA | * 23,354,381- | 0.6 μl | 10 μM |
| | | | AGTATA | 23,354,402 | | |
| | | | (SEQ ID NO.: 137) | | | |
| GSDR- | HLA-DRB | 5′ amp primer | TGTAAAACGACGGCCAGTCGTTTCTTGGAGTACT | * 23,354,383- | 0.6 μl | 10 μM |
| 08/12 | | | CTABGGG | 23,354,405 | | |
| | | | (SEQ ID NO.: 138) | | | |
| GSDR- | HLA-DRB | 5′ amp primer | TGTAAAACGACGGCCAGTTTTCTTGGAGTACTCT | * 23,354,383- | 0.6 μl | 10 μM |
| 08/12c | | | ABGGG | 23,354,403 | | |
| | | | (SEQ ID NO.: 139) | | | |
| GSDR- | HLA-DRB | 5′ amp primer | TGTAAAACGACGGCCAGTGTTTCTTGGAGTACTC | * 23,354,382- | 0.6 μl | 10 μM |
| 08/12d | | | TABGGGT | 23,354,404 | | |
| | | | (SEQ ID NO.: 140) | | | |
| GSDR- | HLA-DRB | 5′ amp primer | TGTAAAACGACGGCCAGTTTTCTTGGAGTACTCT | * 23,354,382- | 0.6 μl | 10 μM |
| 08/12e | | | ABGGGT | 23,354,405 | | |
| | | | (SEQ ID No.: 141) | | | |
| GRDR-09- | HLA-DRB | 5′ amp primer | TGTAAAACGACGGCCAGTGTTTCTTGAAGCAGGA | * 23,354,383- | 0.6 μl | 10 μM |
| 2 | | | TAAGTT | 23,354,404 | | |
| | | | (SEQ ID NO.: 142) | | | |
| GSDR-10 | HLA-DRB | 5′ amp primer | TGTAAAACGACGGCCAGTCACAGCACGTTTCTTG | * 23,354,393- | 0.6 μl | 10 μM |
| | | | GAGG | 23,354,412 | | |
| | | | (SEQ ID NO.: 143) | | | |
| GSDR-B3 | HLA-DRB | 5′ amp primer | TGTAAAACGACGGCCAGTGSAGCTGYKTAAGTCT | * 23,290,388- | 0.6 μl | 10 μM |
| | | | GAGT | 23,290,407 | | |
| | | | (SEQ ID NO.: 144) | | | |
| GSDR-B4 | HLA-DRB | 5′ amp primer | TGTAAAACGACGGCCAGTAGCGAGTGTGGAACCT | *** 8,780- | 0.6 μl | 10 μM |
| | | | GATC | 8,799 | | |
| | | | (SEQ ID NO.: 145) | | | |
| GSDR-B5 | HLA-DRB | 5′ amp primer | TGTAAAACGACGGCCAGTGCAGCAGGATAAGTAT | **** | 0.6 μl | 10 μM |
| | | | GA | 23,348,211- | | |
| | | | (SEQ ID NO.: 146) | 23,348,229 | | |
| GSDR-3′ | HLA-DRB | 3′ amp primer | CAGGAAACAGCTATGACCGCTYACCTCGCCKCTG | * 23,354,132- | 0.6 μl | 10 μM |
| Universal | | | CAC | 23,354,150 | | |
| | | | (SEQ ID NO.: 147) | | | |
| CRP 1 | HLA-DRB | 5′ amp primer | TCATGCTTTTGGCCAGACAG | ** 18,067- | 0.25 μl | 10 μM |
| | | | (SEQ ID NO.: 148) | 18,086 | | |
| CRP 3 | HLA-DRB | 3′ amp primer | GGCGGACTCCCAGCTTGTA | ** 18,650- | 0.25 μl | 10 μM |
| | | | (SEQ ID NO.: 149) | 18,668 | | |
| yDR86- | HLA-DRB | seq primer | CTGCACYGTGAAKCTCTCCA | * 23,354,145- | 1 μl | 3 μM |
| TG-1 | Codon86-GTG | | (SEQ ID NO.: 150) | 23,354,164 | | |
| yDR86- | HLA-DRB | seq primer | GCACYGTGAAKCTCTCCAC | * 23,354,147- | 1 μl | 3 μM |
| TG-13 | Codon86-GTG | | (SEQ ID NO.: 151) | 23,354,165 | | |
| yDR86- | HLA-DRB | seq primer | GCACYGTGAAGCTCTCACC | * 23,354,147- | 1 μl | 3 μM |
| GT-13 | Codon86-GGT | | (SEQ ID NO.: 152) | 23.354,165 | | |
| yDR86- | HLA-DRB | seq primer | TTTTTTTTTTTTTTGCACYGTGAAGCTCTTACC | * 23,354,147- | 1 μl | 3 μM |
| GT-13Ta | Codon86-GGT | | (SEQ ID NO.: 153) | 23,354,165 | | |
| yDR86- | HLA-DRB | seq primer | TTTTTTTTTTTTTTGTACYGTGAAKCTCCCCAC | * 23,354,147- | 1 μl | 3 μM |
| GT-13Tb | Codon86-GTG | | (SEQ ID NO.: 154) | 23,354,165 | | |
| yDR86- | HLA-DRB | seq primer | TTTTTTTTTTTTTTGCACYGTGAAKCTCCCCAC | * 23,354,147- | 1 μl | 3 μM |
| GT-13Tc | Codon86-GTG | | (SEQ ID NO.: 155) | 23,354,165 | | |
| yDR86- | HLA-DRB | seq primer | TTTTTTTTTTTTTTGTACYGTGAAKCTCACCAC | * 23,354,147- | 1 μl | 3 μM |
| GT-13Td | Codon86-GTG | | (SEQ ID NO.: 156) | 23,354,165 | | |
| yDR86- | HLA-DRB | seq primer | TTTTTTTTTTTTTTGCACYGTGAAKCTCACCAC | * 23,354,147- | 1 μl | 3 μM |
| GT-13Te | Codon86-GTG | | (SEQ ID NO.: 157) | 23,354,165 | | |
| M13 | | seq primer | TGTAAAACGACGGCCAGT | N/A | 1 μl | 3 μM |
| Forward | | | (SEQ ID NO.: 131) | | | |
| M13 | | seq primer | CAGGAAACAGCTATGACC | N/A | 1 μl | 3 μM |
| Reverse | | | (SEQ ID NO.: 132) | | | |
| yGSDR-07 | HLA-DRB | seq primer | CTGTGGCAGGGTAAGTATA | * 23,354,381- | 1 μl | 3 μM |
| | | | (SEQ ID NO.: 158) | 23,354,399 | | |
| yGSDR-04 | HLA-DRB | seq primer | TTCTTGGAGCAGGTTAAAC | * 23,354,384- | 1 μl | 3 μM |
| | | | (SEQ ID NO.: 159) | 23,354,402 | | |
| yGSDR-02 | HLA-DRB | seq primer | CCTGTGGCAGCCTAAGA | * 23,354,384- | 1 μl | 3 μM |
| | | | (SEQ ID NO.: 160) | 23,354,400 | | |
| yGSDR-01 | HLA-DRB | seq primer | CGTTTCTTGTGGSAGCTT | * 23,354,388- | 1 μl | 3 μM |
| | | | (SEQ ID NO.: 161) | 23,354,405 | | |
| yGSDR- | HLA-DRB | seq primer | CTTGTGGSAGCT | * 23,354,393- | 1 μl | 3 μM |
| 01g | | | (SEQ ID No.: 219) | 23,354,404 | | |
| yGSDR- | HLA-DRB | seq primer | TTCTTGGAGTACTCTACGTC | * 23,354,388- | 1 μl | 3 μM |
| 03/5/6 | | | (SEQ ID NO.: 162) | 23,354,402 | | |
| yGSDR- | HLA-DR | seq primer | CGTTTCTTGGAGTACTCTACGTC | * 23,354,385- | 1 μl | 3 μM |
| 03/5/6a | | | (SEQ ID No. 220) | 23,354,402 | | |
| yGSDR-07 | HLA-DRB | seq primer | CCACAGCACGTTTCTTGTG | * 23,354,395- | 1 μl | 3 μM |
| | | | (SEQ ID NO.: 163) | 23,354,413 | | |
| yGSDR- | HLA-DRB | seq primer | CGTTTCTTGGAGTACTCTACGGG | * 23,354,383- | 1 μl | 3 μM |
| 08/12 | | | (SEQ ID NO.: 164) | 23,354,405 | | |
| yGSDR- | HLA-DRB | seq primer | GTTTCTTGGAGTACTCTABGGGT | * 23,354,388- | 1μl | 3 μM |
| 08/12-o | | | (SEQ ID No.: 221) | 23,354,406 | | |

DP Locus Single Tube Multiplex Primers

| | | | | | | |
|---|---|---|---|---|---|---|
| DPB1F1 | HLA-DP | amp primer | TGTAAAACGACGGCCAGTCCTCCCCGCAGAGAAT | * 23,845,597- | 0.6 μl | 5 μM |
| | | | TAMGTG | 23,845,618 | | |
| | | | (SEQ ID NO.: 165) | | | |
| DPB1F2 | HLA-DP | amp primer | TGTAAAACGACGGCCAGTCCTCCCCGCAGAGAAT | *23,845,597- | 0.6 μl | 5 μM |
| | | | TACCTT | 23,845,618 | | |
| | | | (SEQ ID NO.: 166) | | | |
| DPB1R1 | HLA-DP | amp primer | CAGGAAACAGCTATGACCGCGCTGYAGGGTCACG | *23,845,848- | 0.6 μl | 5 μM |
| | | | GCCT | 23,845,867 | | |
| | | | (SEQ ID NO.: 167) | | | |
| DPB1R2 | HLA-DP | amp primer | CAGGAAACAGCTATGACCGCGCTGCAGGGTCATG | *23,845,848- | 0.6 μl | 5 μM |
| | | | GGCC | 23,845,867 | | |
| | | | (SEQ ID NO.: 168) | | | |
| CRP1 | HLA-DP | seq primer | TCATGCTTTTGGCCAGACAG | ** 18,067- | 0.2 μl | 10 μM |
| | | | (SEQ ID NO.: 148) | 18,086 | | |
| CRP3 | HLA-DP | seq primer | GGCGGACTCCCAGCTTGTA | ** 18,650- | 0.2 μl | 10 μM |
| | | | (SEQ ID NO.: 149) | 18,668, | | |
| M13 Forward | | seq primer | TGTAAAACGACGGCCAGT | N/A | 1 μl | 3 μM |
| | | | (SEQ ID NO.: 131) | | | |
| M13 Reverse | | seq primer | CAGGAAACAGCTATGACC | N/A | 1 μl | 3 μM |
| | | | (SEQ ID NO.: 132) | | | |

DQ Locus Single Tube Multiplex Primers

| | | | | | | |
|---|---|---|---|---|---|---|
| DQInt1T | HLA-DQ | amp primer | TGTAAAACGACGGCCAGTGGTGATTCCCCGGAGA | * 23,429,522- | 0.25 μl | 25 μM |
| | | | GGAT | 23,429,541 | | |
| | | | (SEQ ID NO.: 169) | | | |
| DQInt1G | HLA-DQ | amp primer | TGTAAAACGACGGCCAGTATTCCYCGCAGAGGAT | * 23,429,526- | 0.7 μl | 10 μM |
| | | | TTCG | 23,429,545 | | |
| | | | (SEQ ID No.: 222) | | | |
| DQBIN2R-11 | HLA-DQ | amp primer | CAGGAAACAGCTATGACCGGGCCTCGCAGASGGG | * 23,429,228- | 0.08 μl | 25 μM |
| | | | CGACG | 23,429,248 | | |
| | | | (SEQ ID NO.: 170) | | | |
| DQBIN2R-11- | HLA-DQ | amp primer | CAGGAAACAGCTATGACCGGCGACGCCGCTCACC | * 23,429,217- | 0.7 μl | 10 μM |
| 15 | | | TC | 23,429,235 | | |
| | | | (SEQ ID No.: 223) | | | |
| DQBIN2R-12 | HLA-DQ | amp primer | CAGGAAACAGCTATGACCGSGCCTCACGGAGGGG | * 23,429,228- | 0.08 μl | 25 μM |
| | | | CGACG | 23,429,248 | | |
| | | | (SEQ ID NO.: 171) | | | |
| DQBIN2R-12- | HLA-DQ | amp primer | CAGGAAACAGCTATGACCGGCGACGACGCTCACC | * 23,429,217- | 0.7 μl | 10 μM |
| 15 | | | TC | 23,429,235 | | |
| | | | (SEQ ID No.: 224) | | | |
| DQBIN2R-13 | HLA-DQ | amp primer | CAGGAAACAGCTATGACCGCGCCTCACGGAGGGT | * 23,429,228- | 0.08 μl | 25 μM |
| | | | CAACC | 23,429,248 | | |
| | | | (SEQ ID NO.: 172) | | | |
| DQBIN2R-13- | HLA-DQ | amp primer | CAGGAAACAGCTATGACCGTCAACCACGCTCACC | * 23,429,217- | 0.7 μl | 10 μM |
| 15 | | | TC | 23,429,235 | | |
| | | | (SEQ ID No.: 225) | | | |
| DQX3 Forward | HLA-DQ | amp primer | CAGTCGAGGCTGATAGCGAGCTCCCTGTCTGTTA | * 23,426,360- | 0.7 μl | 10 μM |
| Amp | | | CTGCCCTYAG | 23,426,390 | | |
| | | | (SEQ ID NO.: 173) | | | |
| DQX3FAP3 + 1 | HLA-DQ | amp primer | CAGTCGAGGCTGATAGCGAGCTCCCCTGTCTGTT | * 23,426,360- | 0.2 ∥l | 10 μM |
| | | | ACTGCCCTCAG | 23,426,390 | | |
| | | | (SEQ ID No.: 226) | | | |
| DQX3 Reverse | HLA-DQ | amp primer | CTATCAACAGGTTGAACTGGGCCCACAGTAACAG | * 23,426,053- | 0.7 μl | 10 μM |
| Amp 1 | | | AAACTCAATA | 23,426,077 | | |
| | | | (SEQ ID NO.: 174) | | | |
| DQX3 Reverse | HLA-DQ | amp primer | CTATCAACAGGTTGAACTGGGCCCATAATAACAG | * 23,426,053- | 0.7 μl | 10 μM |
| Amp2 | | | AAACTCAATA | 23,426,077 | | |
| | | | (SEQ ID NO.: 175) | | | |
| DQX3FAP4 + 1 | HLA-DQ | amp primer | CAGTCGAGGCTGATAGCGAGCTCCCCTGTCTGTT | * 23,426,360- | 0.2 μl | 10 μM |
| | | | ACTGCCCTTAG | 23,426,390 | | |
| | | | (SEQ ID No.: 227) | | | |
| DQX3FAP5 + 1 | HLA-DQ | amp primer | CAGTCGAGGCTGATAGCGAGCTTTCCTGTCTGTT | * 23,426,360- | 0.3 μl | 10 μM |
| | | | ACTGCCCTTAG | 23,426,390 | | |
| | | | (SEQ ID No.: 228) | | | |
| DQX3FAPa + 1 | HLA-DQ | amp primer | CAGTCGAGGCTGATAGCGAGCTCTCCTGTCTGTT | * 23,426,360- | 0.2 μl | 10 μM |
| | | | ACTGCCCTGAG | 23,426,390 | | |
| | | | (SEQ ID No.: 229) | | | |
| DQX3RAP3c | HLA-DQ | amp primer | CTATCAACAGGTTGAACTGGGCCCATAGTAACAG | * 23,426,053- | 0.35 μl | 10 μM |
| | | | AAACTCAATA | 23,426,077 | | |
| | | | (SEQ ID No.: 230) | | | |
| DQ Int1-3 | HLA-DQ | amp primer | CAGGAAACAGCTATGACCACTGACTGGCCGGTGA | *23,429,533- | 0.5 μl | 10 μM |
| | | | TTCC | 23,429,552 | | |
| | | | (SEQ ID NO.: 176) | | | |
| DQ Int1-4 | HLA-DQ | amp primer | CAGGAAACAGCTATGACCACTGACCGGCCGGTGA | * 23,429,533- | 0.5 μl | 10 μM |
| | | | TTCC | 23,429,522 | | |
| | | | (SEQ ID NO.: 177) | | | |
| DQBIN2R-4 | HLA-DQ | amp primer | GTAAAACGACGGCCAGTATGGGCCTCGCAGACGG | * 23,429,226- | 0.5 μl | 10 μM |
| | | | GCGACGA | 23,429,249 | | |
| | | | (SEQ ID NO.: 178) | | | |
| DQBIN2R-5 | HLA-DQ | amp primer | CAGGAAACAGCTATGACCCCTGCCCCCACCACTC | * 23,429,111- | 0.5 μl | 10 μM |
| | | | TCGC | 23,429,130 | | |
| | | | (SEQ ID NO.: 179) | | | |
| DQBIN2R-6 | HLA-DQ | amp primer | CAGGAAACAGCTATGACCGACACTAGGCAGCCTG | * 23,429,041- | 0.5 μl | 10 μM |
| | | | GCCAA | 23,429,062 | | |
| | | | (SEQ ID NO.: 180) | | | |
| DQBIN2R-7 | HLA-DQ | amp primer | CAGGAAACAGCTATGACCCAGAGCAGAGGACAAG | * 23,429,002- | 0.5 μl | 10 μM |
| | | | GCCGACG | 23,429,024 | | |
| | | | (SEQ ID NO.: 181) | | | |
| DQBIN2R-8 | HLA-DQ | amp primer | CAGGAAACAGCTATGACCAAAAGGAGGCAAATGC | * 23,428,963- | 0.5 μl | 10 μM |
| | | | ATAAGGCACG | 23,428,988 | | |
| | | | (SEQ ID NO.: 182) | | | |
| DQBIN2R-9 | HLA-DQ | amp primer | CAGGAAACAGCTATGACCGCGCCTCACGGAGGGG | * 23,429,228- | 0.5 μ | 10 μM |
| | | | CGACGA | 23,429,249 | | |
| | | | (SEQ ID NO.: 183) | | | |
| DQBIN2R-10 | HLA-DQ | amp primer | GTAAAACGACGGCCAGTGGGCCTCGCAGAGGGGC | * 23,429,228- | 0.5 μl | 10 μM |
| | | | GACGC | 23,429,249 | | |
| | | | (SEQ ID NO.: 184) | | | |
| Reverse Seq | HLA-DQ | seq primer | CTATCAACAGGTTGAACTG | N/A | 1 μl | 3 μM |
| Primer | | | (SEQ ID NO.: 185) | | | |
| Forward Seq | HLA-DQ | seq primer | CAGTCGAGGCTGATAGCGAGCT | N/A | 1 μl | 3 μM |
| Primer | | | (SEQ ID NO.: 186) | | | |
| M13 Forward | | seq primer | TGTAAAACGACGGCCAGT | N/A | 1 μl | 3 μM |
| | | | (SEQ ID NO.: 131) | | | |
| M13 Reverse | | seq primer | CAGGAAACAGCTATGACC | N/A | 1 μl | 3 μM |
| | | | (SEQ ID NO.: 132) | | | |

DQ Locus Multiple Tube Multiplex Primers

| | | | | | | |
|---|---|---|---|---|---|---|
| DQ2M13uni | HLA-DQ | amp primer | GTAAAACGACGGCCAGTGCGTGCGTCTTGTGAGC | * 23,429,451- | 0.25 μl | 25 μM |
| | | | AGAAG | 23,429,472 | | |
| | | | (SEQ ID NO.: 187) | | | |
| DQ3M13uni | HLA-DQ | amp primer | GTAAAACGACGGCCAGTGTGCTACTTCACCAACG | * 23,429,477- | 0.25 μl | 25 μM |
| | | | GGAGG | 23,429,498 | | |
| | | | (SEQ ID NO.: 188) | | | |
| DQ4M13uni | HLA-DQ | amp primer | GTAAAACGACGGCCAGTGTGCTACTTCACCAACG | * 23,429,477- | 0.25 μl | 25 μM |
| | | | GGAGC | 23,429,498 | | |
| | | | (SEQ ID NO.: 189) | | | |
| DQ234M13rev | HLA-DQ | amp primer | CAGGAAACAGCTATGACCTCGCCGCTGCAAGGTC | * 23,429,258- | 0.25 μl | 25 μM |
| | | | GT | 23,429,275 | | |
| | | | (SEQ ID NO.: 190) | | | |
| DQ5M13uni | HLA-DQ | amp primer | GTAAAACGACGGCCAGTGATTTCGTGTACCAGTT | * 23,429,500- | 0.25 μl | 25 μM |
| | | | TAAGGGTC | 23,429,524 | | |
| | | | (SEQ ID NO.: 191) | | | |
| DQ6AM13uni | HLA-DQ | amp primer | GTAAAACGACGGCCAGTAGGATTTCGTGTACCAG | * 23,429,500- | 0.25 μl | 25 μM |
| | | | TTTAAGGGTA | 23,429,526 | | |
| | | | (SEQ ID NO.: 192) | | | |
| DQ6TAM13uni | HLA-DQ | amp primer | GTAAAACGACGGCCAGTAGGATTTCGTGTTCCAG | * 23,429,500- | 0.25 μl | 25 μM |
| | | | TTTAAGGGTA | 23,429,526 | | |
| | | | (SEQ ID NO.: 193) | | | |
| DQ6TCAM13uni | HLA-DQ | amp primer | GTAAAACGACGGCCAGTAGGATTTCGTGTTCCAG | * 23,429,500- | 0.25 μl | 25 μM |
| | | | TTTAAGGCTA | 23,429,526 | | |
| | | | (SEQ ID NO.: 194) | | | |
| DQ1AM13Rev | HLA-DQ | amp primer | CAGGAAACAGCTATGACCTCTCCTCTGCAAGATC | * 23,429,258- | 0.25 μl | 25 μM |
| | | | CC | 23,429,275 | | |
| | | | (SEQ ID NO.: 195) | | | |
| DQ1BM13Rev | HLA-DQ | amp primer | CAGGAAACAGCTATGACCTCTCCTCTGCAGGATC | * 23,429,258- | 0.25 μl | 25 μM |
| | | | CC | 23,429,275 | | |
| | | | (SEQ ID NO.: 196) | | | |
| DQX3 Forward | HLA-DQ | amp primer | CAGTCGAGGCTGATAGCGAGCTCCCTGTCTGTTA | * 23,426,369- | 0.7 μl | 10 μM |
| Amp | | | CTGCCCTYAG | 23,426,390 | | |
| | | | (SEQ ID NO.: 173) | | | |
| DQX3 Reverse | HLA-DQ | amp primer | CTATCAACAGGTTGAACTGGGCCCACAGTAACAG | * 23,426,053- | 0.7 μl | 10 μM |
| Amp 1 | | | AAACTCAATA | 23,426,077 | | |
| | | | (SEQ ID NO.: 174) | | | |
| DQX3 Reverse | HLA-DQ | amp primer | CTATCAACAGGTTGAACTGGGCCCATAATAACAG | * 23,426,053- | 0.7 μ1 | 10 μM |
| Amp 2 | | | AAACTCAATA | 23,426,077 | | |
| | | | (SEQ ID NO.: 175) | | | |
| Reverse Seq | HLA-DQ | seq primer | CTATCAACAGGTTGAACTG | N/A | 1 μl | 3 μM |
| Primer | | | (SEQ ID NO.: 185) | | | |
| Forward Seq | HLA-DQ | seq primer | CAGTCGAGGCTGATAGCGAGCT | N/A | 1 μl | 3 μM |
| Primer | | | (SEQ ID NO.: 186) | | | |
| M13 Forward | | seq primer | TGTAAAACGACGGCCAGT | N/A | 1 μl | 3 μM |
| | | | (SEQ ID NO.: 131) | | | |
| M13 Reverse | | seq primer | CAGGAAACAGCTATGACC | N/A | 1 μl | 3 μM |
| | | | (SEQ ID NO.: 132) | | | |

DQ Locus Potential Group Multiplex Sequencing Primers

| | | | | | | |
|---|---|---|---|---|---|---|
| yDQ2 | HLA-DQ | seq primer | GTGCGTCTTGTGAGCAGAAG | * 23,429,451- | 1 μl | 3 μM |
| | | | (SEQ ID NO.: 197) | 23,429,470 | | |
| yDQ3 | HLA-DQ | seq primer | GCTACTTCACCAACGGGAGG | * 23,429,477- | 1 μl | 3 μM |
| | | | (SEQ ID NO.: 198) | 23,429,496 | | |
| yDQ4 | HLA-DQ | seq primer | GCTACTTCACCAACGGGAGC | * 23,429,477- | 1 μl | 3 μM |
| | | | (SEQ ID NO.: 199) | 23,429,496 | | |
| yDQ5 | HLA-DQ | seq primer | TTCGTGTACCAGTTTAAGGGTC | * 23,429,500- | 1 μl | 3 μM |
| | | | (SEQ ID NO.: 200) | 23,429,521 | | |
| yDQ6A | HLA-DQ | seq primer | ATTTCGTGTACCAGTTTAAGGGTA | * 23,429,500- | 1 μl | 3 μM |
| | | | (SEQ ID NO.: 201) | 23,429,523 | | |
| yDQ6TA | HLA-DQ | seq primer | ATTTCGTGTTCCAGTTTAAGGGTA | * 23,429,500- | 1 μl | 3 μM |
| | | | (SEQ ID NO.: 202) | 23,429,523 | | |
| yDQ6TCA | HLA-DQ | seq primer | ATTTCGTGTTCCAGTTTAAGGCTA | * 23,429,500- | 1 μl | 3 μM |
| | | | (SEQ ID NO.: 203) | 23,429,523 | | |

Location as compared to sequence of:
* Reference Accession # NT_007592.13
** Reference Accession # AF4428 18.1
*** Reference Accession # NG_002433.1
**** Reference Accession # NT_007592.14

Exemplary embodiments of the present primers and methods for amplifying and sequencing HLA alleles are provided in the following examples. The following examples are presented to illustrate the methods and to assist one of ordinary skill in using the same. The examples are not intended in any way to otherwise limit the scope of the invention.

### EXAMPLES

The following examples illustrate primer pairs, primer sets and amplification and sequencing methods in accordance with the present invention. In each example PCR was used in the amplification protocol. Unless otherwise provided, the PCR protocol was conducted as described herein. Primer validation was achieved by comparing allele identity derived from using the current primers to previously typed samples available from official cell line repositories such as the UCLA cell line collection and the International Histocompatibility Workshop (IHW) cell line collection. The cell lines used to validate the primers are all previously sequence based typed international reference lines and are used repeatedly for proficiency testing in many clinical HLA typing labs.

In each PCR amplification, a target nucleic acid sample was mixed with a “master mix” containing the reaction components for performing an amplification reaction and the resulting reaction mixture was subjected to temperature conditions that allowed for the amplification of the target nucleic acid. The reaction components in the master mix included a 10×PCR buffer which regulates the pH of the reaction mixture, magnesium chloride (MgCl₂), deoxynucleotides (dATP, dCTP, dGTP, dTTP—present in approximately equal concentrations), that provide the energy and nucleosides necessary for the synthesis of DNA, DMSO, primers or primer pairs that bind to the DNA template in order to facilitate the initiation of DNA synthesis and Thermus aquaticus (Taq) polymerase. Although Taq polymerase was used in the present amplification methods, any suitable polymerase can be used. Generally, preferred polymerases for use with the present invention have low error rates.

More particularly, the reaction components used in the master mix contained a 10×PCR buffer that had been brought down to between a 0.5× and 2.0× concentration (typically 1×) in the reaction, and had an MgCl₂ concentration between about 1.0 and 2.5 mM. Typically, an MgCl₂ concentration of 2.0 mM was used for single tube amplifications and an MgCl₂ concentration of 2.5 mM was used for group specific amplifications. The dNTPs in the master mix were brought to a concentration of about 0.5 to 2% (typically 1%) in the reaction, and the DMSO was used at a concentration of about 5 to 15% (typically about 8%). The primer concentration in each PCR amplification ranged from about 10 to 30 pmol/μl.

In the polymerase chain reactions, the thermal cycling reaction used in DNA amplification had a temperature profile that involved an initial ramp up to a predetermined, target denaturation temperature that was high enough to separate the double-stranded target DNA into single strands. Generally, the target denaturation temperature of the thermal cycling reaction was approximately 91-97° C. and the reaction was held at this temperature for a time period ranging between 20 seconds to fifteen minutes. Then, the temperature of the reaction mixture was lowered to a target annealing temperature which allowed the primers to anneal or hybridize to the single strands of DNA. The annealing temperatures ranged from 45° C.-74° C. depending on the sequence sought to be amplified. Next, the temperature of the reaction mixture was raised to a target extension temperature to promote the synthesis of extension products. The extension temperature was held for approximately two minutes and occurred at a temperature range between the annealing and denaturing temperatures. This completed one cycle of the thermal cycling reaction. The next cycle started by raising the temperature of the reaction mixture to the denaturation temperature. The cycle was repeated 10 to 35 times to provide the desired quantity of DNA. Substantially similar amplification reaction conditions include conditions where the primer concentration, Mg²⁺ concentration, salt concentration and annealing temperature remain static.

The resulting PCR data had a background of less than 20% of the overall signal and less than a 30% difference in the evenness of the peaks. The average signal strength was between about 100 and 4000 units, however excessive background resulted for signals above about 2000 when the samples were sequenced using an ABI 377 automatic sequencer. Full sequences of the exons of interest were be readable from beginning to end as a result of the sequencing reaction.

### Example 1

### Amplification of Alleles of A, B and DR Loci

This example demonstrates the use of the present primer pairs and primer sets in non-multiplex and multiplex amplification of HLA alleles of the A, B and DR loci. In each instance, the primers were used in the PCR protocol outlined above.

### A. A Locus Non-Multiplex Amplification

Amplification Primers: The single 5′ primer (pA5-3) begins in the A Locus 5′ untranslated region and ends in exon 1. The single 3′ (pA3-29-2) primer is in exon 5. This is a locus specific amplification and all alleles in the A locus are amplified with this primer set.

Sequencing Primers: All sequencing primers, including three forward sequencing primers and three reverse sequencing primers are located in the introns flanking exons 2, 3 and 4 (Aex2F, Aex2R-4, Aex3F-2, Aex3R-3, Aex4F, and Aex4R-5). The multiplexing of the sequencing primers allows bi-directional sequencing of exons 2, 3 and 4.

### B. B Locus Multiplex Amplification

Amplification Primers: Three 5′ primers in exon 1, a C primer (pB5-48a) and two G primers (pB5-49+1Ca and pB5-49+1A). There is one 3′ intron 3 primer (pB3-24) for amplification of the exon 2-exon 3 product. The alleles are segregated by the presence of a G or C at a defined base in exon 1. Approximately half of the alleles have a C at that position, the other half a G. The alleles in the B Locus, which are labeled according to convention known in the art are divided roughly in half between the two primers in exon 1 as follows in Table 2:

TABLE 2

| C Group B Locus Alleles | | G Group B Locus Alleles | | |
|---|---|---|---|---|
| 070201 | 380201 | 1301 | 4002 | 5611 |
| 070202 | 390101 | 1302 | 4003 | 570101 |
| 0703 | 390103 | 1303 | 4004 | 5702 |
| 0704 | 390201 | 1304 | 4005 | 570301 |
| 0706 | 390202 | 1308 | 400601 | 5706 |
| 0709 | 3903 | 180101 | 400602 | 5801 |
| 0718 | 3904 | 1802 | 4008 | 5802 |
| 0801 | 3905 | 1803 | 4013 | 5804 |
| 0802 | 390601 | 1806 | 4020 | 5901 |
| 1401 | 390602 | 2702 | 44020101 | 7801 |
| 1402 | 3908 | 2703 | 44020102S | 780201 |
| 1405 | 3909 | 2704 | 44301 | 8101 |
| 15010101 | 3910 | 270502 | 440302 | 8202 |
| 1502 | 3917 | 270504 | 4404 | 8301 |
| 1503 | 3924 | 270505 | 406 | |
| 1508 | 400101 | 2706 | 4407 | |
| 1509 | 400102 | 2708 | 4408 | |
| 1510 | 4007 | 2709 | 4409 | |
| 151101 | 4012 | 2711 | 4413 | |
| 151102 | 4016 | 2712 | 4431 | |
| 1512 | 4023 | 2713 | 47010101 | |
| 1513 | 4101 | 2714 | 47010102 | |
| 1514 | 4102 | 2718 | 4702 | |
| 1515 | 4201 | 350101 | 510101 | |
| 1516 | 4418 | 3502 | 510102 | |
| 151701 | 4501 | 3503 | 510105 | |
| 151702 | 4504 | 3504 | 510201 | |
| 1518 | 4601 | 3505 | 510202 | |
| 1519 | 4801 | 3506 | 5103 | |
| 1520 | 4802 | 3507 | 5104 | |
| 1521 | 4805 | 3508 | 5108 | |
| 1523 | 4901 | 3511 | 520101 | |
| 1525 | 5001 | 3512 | 520102 | |
| 1528 | 5002 | 3515 | 5204 | |
| 1529 | 670101 | 3528 | 5301 | |
| 1546 | 6702 | 3531 | 5401 | |
| 1552 | 7301 | 3541 | 5501 | |
| 1553 | | 3542 | 5502 | |
| 1554 | | 3543 | 5505 | |
| 1555 | | 3701 | 5512 | |
| 1557 | | 3702 | 5601 | |
| 1558 | | 3704 | 5602 | |
| 1566 | | 3705 | 5603 | |

There is one 5′ inton 3 primer (pB5-55+4) and four 3′ primers (pB3-20, pB3-21, pB3-22 and pB3-23) in exon 5 for amplification of the exon 4 product (primers are multiplexed to cover the complexity of B Locus in this exon). Thus, these primers anneal to four distinct sequences. In order to amplify all of the known alleles in HLA Locus B, each of the four primers was included in a cocktail of reverse primers. In some embodiments, each 5′ primer will be amplified with the cocktail of 3′ primers in individual reaction tubes.

Sequencing Primers: All sequencing primers are located in the introns flanking exons 2, 3 and 4 (yB2F-6a+10, yB2F-6b+10, yB2F-6c+10, yB2F-5a+10, yB2F-5b+10, yB2F-5c+10, yB2F-12a+10, yB2F-12b+10, yB2F-12c+10, yB2F-19b+10, yB2F-19c+10, yB2R-4, yB3F-2a+10, yB3F-2b+10, yB3F-2c+10, B-Ex3R, B-Ex4F1, and yB4R-3). The sequencing primers include at least one forward and one reverse sequencing primer for each primer location.

### C. DRB1 Single Tube Multiplex Amplification

Amplification Primers: There are six 5′ amplification primers that begin in intron 1 and end in exon 2 (OTDR-01, OTDR-02/07, OTDR-03/5/6/08/12, OTDR-04-5, OTDR-10-4, and OTDR-09-8). Each individual primer is designed to amplify a specific group of alleles at the DRB1 locus: DRB1*01, DRB1*15/16/07, DRB1*03/11/13/14/8/12, DRB1*04, DRB1*09, and DRB1*10. There is one 3′ primer located in exon 2 (OTDR-3-2). All amplification primers are tailed with the M13 sequence. M13 sequence are tails, which do not bind to the HLA allele, that are added to the amplification primers, such as in DR, DQ, and DP that allow the utilization of a single forward and reverse primer during a sequencing reaction irrespective of groups. This results in a reduction in the total number of sequencing primers that must be included in the kit to cover all possible products. The tailing of the amplification primers was also done to increase the resolution and assure full coverage of exon 2 upon sequencing.

Sequencing primers: The sequencing primers are M13 forward (SEQ ID NO.: 131) and M13 reverse (SEQ ID NO.: 132).

### D. DRB1/3/4/5 Multitube Multiplex Amplification

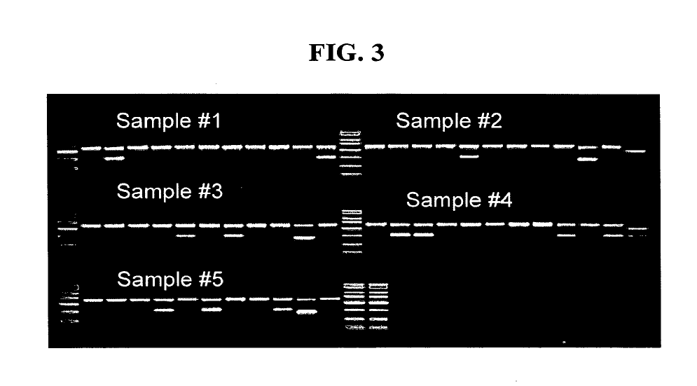

Amplification primers: There are eleven 5′ group specific primers that either begin in intron 1 and end in exon 2 or are fully in exon 2 depending on where the most group specificity exists for the HLA alleles being amplified. Each individual primer is designed to amplify specific alleles at more than one DRB loci: DRB 1*01, DRB1*15/16, DRB1*03/11/13/14, DRB1*04, DRB1*07, DRB1*8/12, DRB1*09, DRB1*10, DRB3, DRB4, DRB5. There is one 3′ primer located in exon 2. Each of the eleven 5′ group specific primers is amplified with the common reverse 3′ primer. All amplification primers are tailed with the M13 sequence. The tailing of the amplification primers was done to assure full coverage of exon 2 upon sequencing. The results of amplification of five individual samples is shown in FIG. 3 (lanes correspond to the specific alleles set forth above). As demonstrated by FIG. 3, the 600 bp product serves as a control. FIG. 3 clearly shows the presence of the particular alleles in the sample.

Sequencing primers: The sequencing primers are M13 forward (SEQ ID NO.: 131) and M13 reverse (SEQ ID NO.: 132). Sequencing confirmed the identity of each allele.

### Example 2

### A and B Locus Multiplex Amplification

This example demonstrates the use of the present primer pairs and primer sets in the multiplex amplification of HLA alleles of the A and B loci. In each instance, the primers were used in the PCR protocol outlined above, using the master mixes shown.

### A. A Locus

| Reagent | Amount |
|---|---|
| Purified water | 9.3 μl |
| 10X PCR Buffer | 2.5 μl |
| Magnesium Chloride | 1.5 μl |
| DMSO | 2.0 μl |
| dNTP (50% deazaG) | 2.5 μl |
| 5′ Primer- pA5-5 | 0.5 μl |
| 3′ Primer- pA3-31 | 0.5 μl |
| 5′ Primer- pA5-3 | 0.5 μl |
| 3′ Primer- pA3-29-2 | 0.5 μl |
| FastStart Taq | 0.2 μl |
| Genomic DNA | 5.0 μl |
| | 25 μl total reaction volume |

### B. B Locus

| Reagent | Amount |
|---|---|
| Purified water | 9.3 μl |
| 10X PCR Buffer | 2.5 μl |
| Magnesium Chloride | 1.5 μl |
| DMSO | 2.0 μl |
| dNTP (50% deazaG) | 2.5 μl |
| 5′ Primer- pB5-48 or 5-49 | 0.5 μl |
| 3′ Primer- pB3-24 | 0.5 μl |
| 5′ Primer- pB5-55 + 4 | 0.5 μl |
| 3′ Primer- pA3-20, 21, 22, 23 | 0.5 μl |
| FastStart Taq | 0.2 μl |
| Genomic DNA | 5.0 μl |
| | 25 μl total reaction volume |

Both A locus and B locus samples were run in a PE 9700 thermal cycler under the following conditions:

| | | | | |
|---|---|---|---|---|
| Initial Denaturation | 95° C. | 4 min | | |
| Denaturation | 95° C. | 20 sec | | |
| Annealing | 63° C. | 20 sec | {close oversize brace} | 35 cycles |
| Extension | 72° C. | 40 sec | | |
| Final Extension | 72° C. | 5 min | | |

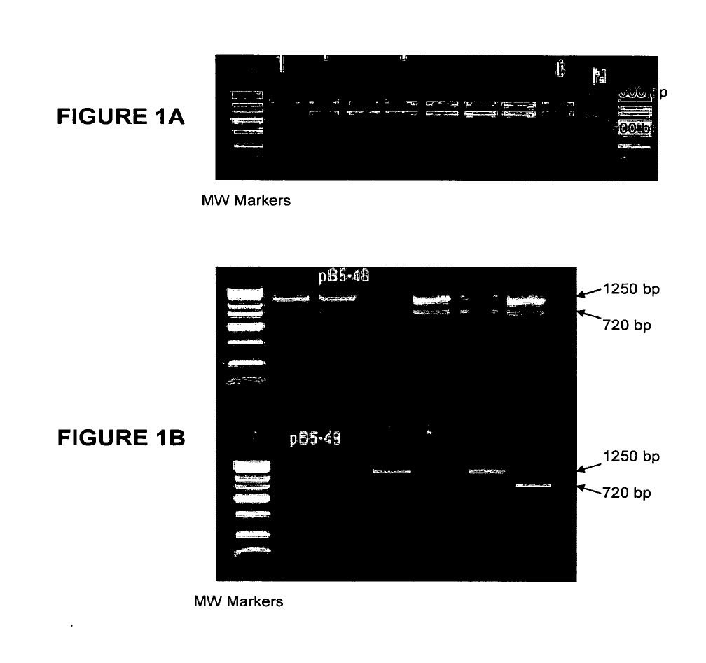

Following amplification, the PCR amplicons were run on a 1.5% agarose gel to check for successful amplification. The results of the A locus agarose gel are demonstrated in FIG. 1A. For the A Locus, the ˜1300 bp band is the product of the amplification using pA5-3+3 and pA3×23b/pA3×23b80 as the primers and the smaller ˜700 bp band is the product of the amplification using pA5-5 and pA3-29-2 as primers. The smaller fragment on the gel acts as a control because of the ability to cross verify that alleles of the correct loci are amplified because the smaller fragment should always be the same at each loci regardless of the allele. The smaller fragment also allows coverage or more of the loci in a smaller fragment thereby producing a more reliable reaction with stronger products and greater flexibility for subsequent incorporation of additional exons. Amplification of a smaller fragment that can serve as a control also allows both a reduction in cycle time and an increase uniformity with other loci (class I and class II). The results of the B locus agarose gel are demonstrated in FIG. 1B. For the B Locus, the ˜1250 bp band is the product of the amplification using pB5-48 or pB5-49 and pB3-24 as primers and the smaller ˜720 bp band is the product of the amplification using pB5-55+4 and pB3-20, pB3-22, and pB3-23 as primers. The smaller amplicon in the HLA B amplification serves the same purposes as the smaller amplicon in the HLA A amplification. In many cases, because the size of the amplicons was so similar between the loci and because the position of the primers on the HLA locus was also similar, agarose gel electrophoresis was used only to check the amplification reaction and not to distinguish between alternative HLA loci. However, in some instances, more sensitive techniques, such as using microfluidic separation may be used to distinguish HLA loci prior to sequencing.

Following confirmation of amplification, to prepare the amplicon for the sequencing reaction, 4 μl of ExoSAP-IT® (USB; Cleveland, Ohio) was added to each amplicon to rid each amplicon of excess primer and dNTPs. Subsequent to the addition of the ExoSAP-IT®, the amplicons were incubated at 37° C. for 20 minutes and then at 80° C. for 20 minutes.

The next step was sequencing of the amplicons. Sequencing reactions for exons 2, 3 and 4 for both HLA A locus and HLA B locus were prepared for each sample using the following mix of reagents:

| | |
|---|---|
| DYEnamic ™ ET Terminators (Amersham | 2 μl |
| Biosciences) | |
| DYEnamic ™ ET Terminator Dilution Buffer | 2 μl |
| Water | 3 μl |
| Sequencing Primer (either forward or reverse) | 1 μl |
| ExoSAP-IT ® treated PCR product | 2 μl |
| | 10 μl total |
| | reaction volume |

Sequencing primers for HLA A consisted of primers Aex2F, Aex2R-4, Aex3F-2, Aex3R-3, Aex4F8001, and Aex4R-5 from Table 1. Sequencing primers for HLA B consisted of primers yB2F-6a+10, yB2F-6b+10, yB2F-6c+10, yB2F-5a+10, yB2F-5b+10, yB2F-5c+10, yB2F-12a+10, yB2F-12b+10, yB2F-12c+10, yB2F-19b+10, yB2F-19c+10, yB2R-4, yB3F-2a+10, yB3F-2b+10, yB3F-2c+10, B-Ex3R, B-Ex4F1, and yB4R-3 from Table 1.

In order to gain sequence analysis, the entire reaction volume of the sequencing reactions were cycled in a PE 9700 thermal cycler under the following conditions:

| | | | |
|---|---|---|---|
| 95° C. | 20 sec | | |
| 50° C. | 15 sec | {close oversize brace} | 25 cycles |
| 60° C. | 60 sec | | |
| 4° C. | Infinite | | |

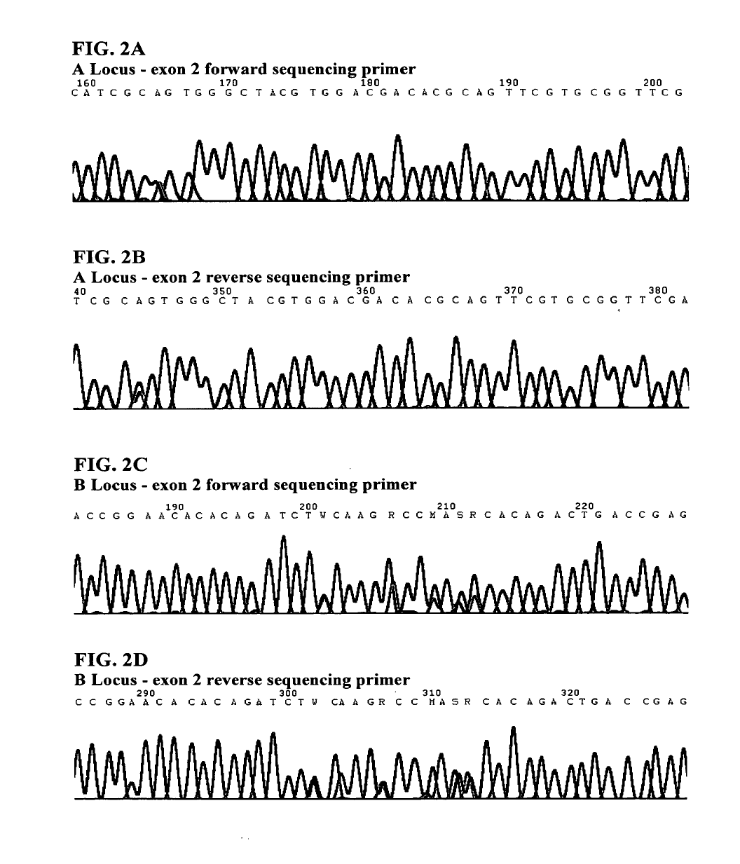

Following completion of the sequencing reaction, ethanol precipitation was used to remove excess terminators and precipitate out the sequencing products. The precipitated products were run on an ABI 3100 capillary sequencer. The electropherogram results of the sequencings reactions are shown in FIGS. 2A-2D.

The present primers and kits can have any or all of the components described herein. Likewise, the present methods can be carried out by performing any of the steps described herein, either alone or in various combinations. One skilled in the art will recognize that all embodiments of the present invention are capable of use with all other appropriate embodiments of the invention described herein. Additionally, one skilled in the art will realize that the present invention also encompasses variations of the present primers, configurations and methods that specifically exclude one or more of the components or steps described herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the invention.

All references, patents and publications disclosed herein are specifically incorporated by reference thereto. Unless otherwise specified, “a” or “an” means “one or more”.

While preferred embodiments have been illustrated and described, it should be understood that changes and modifications can be made therein in accordance with ordinary skill in the art without departing from the invention in its broader aspects as described herein.

1. A primer set comprising:
(a) at least two primers capable of amplifying a portion of all human leukocyte antigen (HLA) alleles of an HLA locus; and
(b) a control primer pair capable of producing an HLA control amplicon of predetermined size by amplifying a portion of a HLA allele only if the HLA locus is present in a sample.

2. The primer set of claim 1 wherein the portion of the HLA allele amplified by the control primer pair is common to all or substantially all HLA alleles.

3. The primer set of claim 1 wherein the portion of the HLA allele amplified by the control primer pair comprises a portion of exon 4 of the HLA A locus or exon 4 of the HLA B locus.

4. The primer set of claim 1 wherein the predetermined size of the HLA control amplicon is about 500 to 1000 base pairs in length.

5. The primer set of claim 1 wherein at least one of the at least two primers has a 5′ portion that is not complementary to the HLA allele.

6. The primer set of claim 5 wherein the 5′ non-complementary portion decreases a melting temperature (Tm) between the primer and a HLA allele, further wherein the decreased melting temperature results in an enhanced specificity of an amplification reaction.

7. The primer set of claim 5 wherein the 5′ non-complementary portion allows for amplification of a more abundant product, further wherein the 5′ portion allows for a more robust amplification reaction.

8. A primer set comprising:
(a) a multiplicity of primers capable of simultaneously amplifying a plurality of a portion of Class I HLA alleles of a HLA locus under a single set of reaction conditions in a multiplex polymerase chain reaction.

9. The primer set of claim 8 wherein the plurality of a portion of Class I HLA alleles belong to a same HLA locus.

10. The primer set of claim 9, wherein the same HLA locus is a HLA A or a HLA B locus.

11. The primer set of claim 8, wherein the multiplicity of primers are capable of producing a first amplicon and a second amplicon from the HLA locus.

12. The primer set of claim 11, wherein the first amplicon spans exon 1 to intron 3 and the second amplicon spans intron 3 to exon 5.

13. The primer set of claim 8 wherein at least one of the multiplicity of primers has a 5′ portion that is not complementary to the portion of the Class I HLA allele.

14. The primer set of claim 13 wherein the 5′ non-complementary portion allows a decrease in a melting temperature (Tm) between the primer and a HLA allele, further wherein the decreased melting temperature results in an enhanced specificity of an amplification reaction.

15. The primer set of claim 13 wherein the 5′ non-complementary portion allows a more abundant product during amplification, further wherein the 5′ portion allows a more robust amplification reaction.

16-28. (canceled)

29. A method for amplifying a class I HLA allele comprising:
(a) performing an amplification reaction on a sample having or suspected of having a Class I HLA allele wherein the amplification reaction utilizes the primer set of claim 8.

30. The method of claim 29 further comprising sequencing any resulting HLA amplicons.

31. The method of claim 29 wherein the sample is a cDNA.

32-40. (canceled)

41. A method for amplifying and detecting the presence of an HLA allele comprising:
(a) amplifying a nucleic acid wherein the amplification reaction comprises at least three primers of claim 8 capable of amplifying all HLA alleles of an HLA locus in a multiplex amplification reaction; and
(b) detecting the presence of the HLA allele.

42. The method of claim 41 wherein detecting the presence of the HLA allele comprises sequencing the amplified nucleic acid in a multiplex sequencing reaction.

43. The method of claim 41 wherein step (a) and step (b) are automated.

44. The method of claim 43 further comprising automation on an array.

45. A kit for amplifying and detecting human leukocyte antigen alleles comprising:
(a) at least two primers capable of amplifying a portion of all human leukocyte antigen (HLA) alleles of an HLA locus; and a control primer pair capable of producing an HLA control amplicon of predetermined size by amplifying a portion of a HLA allele only if the HLA locus is present in a sample; and
(b) at least one primer comprising a 3′ portion and a 5′ portion wherein the 3′ portion is complementary to an HLA allele and the 5′ portion is not complementary to the HLA allele, wherein the primer allows complete resolution of an exonic sequence by a sequencing reaction.
